(19) United States
(12) Patent Application Publication
(10) Pub. No.: US 2009/0093471 A1
(43) Pub. Date: Apr. 9, 2009

(54) Methods of Treating Nervous Disorders

(76) Inventors: Laurent Desire, Paris (FR); Bertrand Leblond, Paris (FR); Eric Beausoleil, Paris (FR); Thierry Taverne, Saint Martin Boulogne-Sur-Mer (FR); Fabien Schweighoffer, Nogent Sur Marne (FR); Virginie Picard, Paris (FR)

Correspondence Address: NIXON & VANDERHYE, PC, 901 NORTH GLEBE ROAD, 11TH FLOOR, ARLINGTON, VA, 22203, US

(21) Appl. No.: 11/989,396

(22) PCT Filed: Jul. 26, 2006
(86) PCT No.: PCT/IB2006/003503

§ 371 Date: Jan. 25, 2008

Related U.S. Application Data
(63) Continuation of application No. 11/190,070, filed on Jul. 27, 2005.

(51) Int. Cl. A61K 31/541; A61K 31/5377; A61K 31/496; A61K 31/4709; G01N 33/566; A61P 25/28

(52) U.S. Cl. 514/227.8; 514/232.8; 514/253.06; 514/314; 435/7.8

(57) ABSTRACT

The invention relates to compositions and methods for treating nervous disorders. More particularly, the invention relates to methods of treating amyloÊd beta peptide-related disorders, particularly Alzheimer's disease, using Rac1 inhibitors. The invention may be used in mammalian subjects, particularly human subjects, at various stages of the disease, including disease onset. The invention also provides methods of producing, identifying, selecting or optimising compounds for use in the treatment of amyloÊd beta peptide-related disorders, based on a determination of the ability of a test compound to inhibit Rac 1.

The invention relates to compositions and methods for treating nervous disorders. More particularly, the invention relates to methods of treating amyloïd beta peptide-related disorders, particularly Alzheimer's disease, using Rac1 inhibitors. The invention may be used in mammalian subjects, particularly human subjects, at various stages of the disease, including disease onset. The invention also provides methods of producing, identifying, selecting or optimising compounds for use in the treatment of amyloïd beta peptide-related disorders, based on a determination of the ability of a test compound to inhibit Rac1.

Alzheimer's disease (AD) is the most common neurodegenerative disorder marked by progressive loss of memory and cognitive ability. The pathology of AD is characterized by the presence of amyloid plaques (Hardy, J. A., and Higgins, G. A. (1992) Science 256, 184-185), intracellular neurofibrillary tangles and pronounced cell death. The β-amyloid peptide (Aβ) (Buxbaum, J. D., Oishi, M., Chen, H. I., Pinkas-Kramarski, R., Jaffe, E. A., Gandy, S. E., and Greengard, P. (1992) Proc. Natl. Acad. Sci. U.S.A. 89, 10075-10078) is the main constituent of senile plaques found in AD brains. Overproduction, intracellular accumulation, aggregation, and deposition in brain of the 42-amino acid form of Aβ (Aβ 42) is associated with early onset, familial AD (Gouras, G. K., Tsai, J., Naslund, J., Vincent, B., Edgar, M., Checker, F., Greenfield, J. P., Haroutunian, V., Buxbaum, J. D., Xu, H., Greengard, P., and Relkin, N. R. (2000) Am. J. Pathol. 156, 15-20). Furthermore, extracellular Aβ 42 appears toxic to neurons in vitro and in vivo (reviewed in Selkoe, D. J. (2001) Physiol. Rev. 81, 741-766). Aβ is generated by proteolysis of an integral membrane protein, the amyloid precursor protein (APP) via at least two post-translational pathways. The amyloidogenic cleavage of APP is a sequential processing of APP initiated by β-secretase (BACE), which cleaves APP within the luminal domain or at the cell surface, generating the N terminus of Aβ (Vassar, R. (2004) J Mol Neurosci 23, 105-114). This cleavage generates several membrane bound proteolytic C-terminal fragments (CTFs), such as the 99 residue β-CTF (also called C99), as well as the secreted APP ectodomain sAPPβ. The C-terminus of Aβ is subsequently generated by intramembraneous cleavage of CTFs by γ-secretase, producing either Aβ40 or Aβ42. The cleavages at residues 40-42 are referred to as γ-cleavage and the cleavage at residues 49-52 are referred to as ε-cleavage (Weidemann, A., Eggert, S., Reinhard, F. B., Vogel, M., Paliga, K., Baier, G., Masters, C. L., Beyreuther, K., and Evin, G. (2002) Biochemistry 41, 2825-2835). The nonamyloidogenic cleavage of APP, which precludes Aβ generation, is mediated by α-secretase, a disintegrin and metalloproteinase 10 (ADAM-10) and ADAM-17, in a reaction believed to occur primarily on the plasma membrane. This proteolytical cleavage by α-secretase occurs within the Aβ region and produces soluble APP (sAPPα), the dominant processing product and the residual membrane bound 10-kDa CTF (CTFα also called C83). Like C99, C83 is a substrate for γ-secretase which cleaves to generate the non amyloidogenic p3 fragment. APP is also a substrate of caspase activities that cleave its cytosolic domain (Weidemann, A., Eggert, S., Reinhard, F. B., Vogel, M., Paliga, K., Baier, G., Masters, C. L., Beyreuther, K., and Evin, G. (2002) Biochemistry 41, 2825-2835; Weidemann, A., Paliga, K., Durrwang, U., Reinhard, F. B., Schuckert, O., Evin, G., and Masters, C. L. (1999) J. Biol. Chem. 274, 5823-5829).

Other nervous disorders are caused or stimulated by Aβ peptides, such as Mild cognitive Impairment (MCI), Down's syndrome, and the like.

WO2004/076445 discloses compounds having anti-proliferative and/or anti-angiogenic activities, as well as their uses for treating various diseases, including cancer and retinopathies.

### SUMMARY OF THE INVENTION

The present invention stems from the discovery that some of the compounds as disclosed in WO2004/076445 modulate the processing of APP, preventing or reducing the production of amyloïd beta peptides Aβ40 and/or Aβ42, thus preventing the formation of insoluble plaques. The present invention also shows that such compounds essentially do not affect Notch cleavage, do not impact sAPPα levels and do not inhibit BACE. The invention further shows that these compounds strongly inhibit Rac1, and implicates, for the first time, Rac-1 in the modulation of APP processing and Aβ generation. The invention thus shows that Rac1 inhibitors represent a new class of molecules for use in the treatment of amyloïd beta peptide-related disorders.

Accordingly, one aspect of the invention relates to a method of treating an amyloïd beta peptide-related disorder in a mammalian subject, comprising administering to a subject in need thereof an amount of a Rac1 inhibitor effective at reducing APP processing in said subject.

A further object of the invention relates to the use of a Rac1 inhibitor for the manufacture of a medicament for treating an amyloïd beta peptide-related disorder in a mammalian subject.

A further aspect of this invention is a method of inhibiting the generation of an amyloïd beta peptide in a mammalian subject, comprising administering to a subject in need thereof an amount of a Rac1 inhibitor effective at reducing APP processing in said subject.

A further object of the invention relates to the use of a Rac1 inhibitor for the manufacture of a medicament for treating an amyloïd beta peptide-related disorder in a mammalian subject by inhibiting the generation of an amyloïd beta peptide in said subject.

A further aspect of this invention is a method of inhibiting the generation of an amyloïd beta peptide in a mammalian subject without substantially altering the Notch cleavage or BACE activity, comprising administering to a subject in need thereof an effective amount of a Rac1 inhibitor.

A further object of this invention relates to a method of treating an amyloïd beta peptide-related disorder in a mammalian subject, comprising administering to a subject in need thereof an amount of a compound of formula (I) as defined below effective at reducing APP processing in said subject.

A further object of the invention relates to the use of a compound of formula (I) as defined below for the manufacture of a medicament for treating an amyloïd beta peptide-related disorder in a mammalian subject.

For use in the present invention, the active compounds may be formulated in the presence of any pharmaceutically acceptable support or excipient, and they may be used either alone or in combination(s), optionally together with any other active agent(s).

The invention may be used to treat various amyloïd beta peptide-related disorders, including Alzheimer's disease, at various stage of the disorder, in any mammalian subject, preferably human subjects.

The invention also relates to a method of producing, identifying, selecting or optimising candidate compounds for use in the treatment of amyloïd beta peptide-related disorders, the method comprising determining whether a test compound inhibits Rac1, Rac1 inhibition being an indication that the test compound is a candidate compound for use in the treatment of amylold beta peptide-related disorders. Rac1 inhibition may be assessed in vitro, ex vivo or in vivo, according to various biological assays which are known per se in the art. Preferably, the compounds are further assessed for their activity towards Notch cleavage, compounds which substantially do not alter Notch cleavage being preferred.

### LEGEND TO THE FIGURES

FIG. 1: Compounds of Formula (I) inhibit Rac1 activation

FIG. 2: Compounds of Formula (I) prevent Aβ 40 and Aβ 42 production in vitro

FIG. 3: Compounds of Formula (I) do not affect BACE and α-secretase pathways

FIG. 4: Compounds of Formula (I) target γ-secretase activity

FIG. 5: Compounds of Formula (I) do not inhibit Notch-1 cleavage

FIG. 6: Compounds of Formula (I) prevent Aβ 40 and Aβ 42 production in vivo

FIG. 7: Rac1 inhibitor prevents Aβ 40 and Aβ 42 production in vitro

### DETAILED DESCRIPTION OF THE INVENTION

Amyloïd Beta Peptide-Related Disorders

The term Amyloïd beta peptide-related disorders include all disorders which are caused or associated with an increase or abnormal production of an Amyloïd beta peptide, particularly of Aβ40 and/or Aβ42. Examples of such disorders include any disease or condition selected from the group consisting of Alzheimer's disease (e.g., for helping prevent or delay the onset of Alzheimer's disease, for helping to slow the progression of Alzheimer's disease, for treating patients with mild cognitive impairment (MCI) and preventing or delaying the onset of Alzheimer's disease in those who would progress from MCI to AD), Down's syndrome, Hereditary Cerebral Hemorrhage with Amyloidosis of the Dutch-Type, cerebral amyloid angiopathy and its potential consequences (e.g., single and recurrent lobar hemorrhages), degenerative dementias, including dementias of mixed vascular and degenerative origin, dementia associated with Parkinson's disease, dementia associated with progressive supranuclear palsy, dementia associated with cortical basal degeneration, or diffuse Lewy body type of Alzheimer's disease.

### Treatment

The terms “treatment” or “treating” include both therapeutic and prophylactic treatment. In particular, the compounds may be used at very early stages of a disease, or before early onset, or after significant progression thereof. The term “treatment” or “treating” designates in particular a reduction of the burden in a patient, such as preventing or delaying the onset of the disease or disease progression, restoring or increasing cognitive functions or memory in a subject, etc.

### Rac1 Inhibitors

Rac1 is a small GTP-binding protein from the Rho family, such as Rho and Cdc42. These small G proteins are activated by GTP/GDP exchange and regulate a wide variety of cellular functions such as gene expression, cytoskeletal reorganization, and vesicle/secretory trafficking. The activated CDC42 or Rac then activates the PAK Ser/Thr kinase family. Recent studies showed the participation of Rho in the formation of stress fibers, while activated Cdc42 induces the formation of filopodia, thin fingerlike extensions containing actin bundles and Rac regulates the formation of lamellipodia or ruffles, curtain-like extensions often formed along the edge of the cell (see Hall, 1998 for review (1998) Science 279, 509-514). In brain, these small G proteins participate in the morphological changes of neurons, localized in growth cones, axons, dendritic trunks, and spines (van Leeuwen, F. N., van Delft, S., Kain, H. E., van der Kammen, R. A., and Collard, J. G. (1999) Nat. Cell Biol. 1, 242-248). In the AD brain, neuronal Cdc42/Rac are upregulated in select neuronal populations in comparison to age-matched controls, in relation to the pathogenic process and neuronal degeneration (Zhu, X., Raina, A. K., Boux, H., Simmons, Z. L., Takeda, A., and Smith, M. A. (2000) Int J. Dev. Neurosci. 18, 433-437). In the mature brain, Rac1, but not Rho nor Cdc42, is present in the raft domain of neuronal membranes (Kumanogoh, H., Miyata, S., Sokawa, Y., and Maekawa, S. (2001) Neurosci. Res. 39, 189-196). In addition, a recent unbiased quantitative proteomics study revealed Rac1 as a raft-associated protein (Foster, L. J., De Hoog, C. L., and Mann, M. (2003) Proc. Natl. Acad. Sci. USA 100, 5813). Other studies showed that activation of Rac1 is associated with its rapid recruitment into the lipid rafts while Cdc42 is not recruited into rafts, but activated by raft-associated moieties 14 and, more important, that Rac1, but not Rho nor Cdc42, regulates the assembly and export to the cell membrane of Golgi-derived lipid rafts (Field, K. A., J. R. Apgar, E. Hong-Geller, R. P. Siraganian, B. Baird, and D. Holowka. (2000) Mol. Biol. Cell 11, 3661; Rozelle, A. L., L. M. Machesky, M. Yamamoto, M. H. Driessens, R. H. Insall, M. G. Roth, K. Luby-Phelps, G. Marriott, A. Hall, and H. L. Yin. 2000 Curr. Biol. 10:311).

Within the context of this invention, the term “Rac1 inhibitor” designates any compound or treatment that reduces or block the activity of Rac1. More preferred Rac1 inhibitors are compounds that inhibit Rac1 activation by its GEFs in an exchange assay, and/or that inhibit Rac1-dependent cytoskeleton rearrangements. Most preferred Rac1 inhibitors are able to divert APP away from γ-secretase cleavage substantially without directly acting as γ-secretase inhibitors. Furthermore, most preferred Rac1 inhibitors are selective over cdc42 and/or RhoA, i.e., do not substantially interact with cdc42 and/or RhoA, respectively.

Particular compounds of this invention are inhibitors of Rac1 B and/or Rac2 and/or Rac3, particularly inhibitors of Rac1 B and Rac2.

In a particular embodiment of this invention, the Rac1 inhibitor is a compound having a general formula (I):

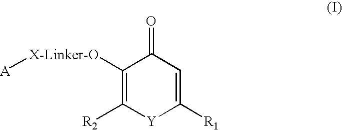

wherein:
R₁ is selected from the group consisting of:

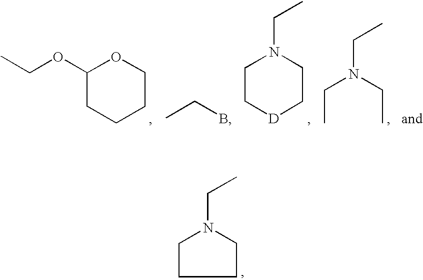

R₂ represents a hydrogen atom, an alkyl or alkenyl group containing from 3 to 6 carbon atoms;
B represents an halogen atom, preferably chlorine or fluorine, a hydroxyl group, a —O—CH₂—O—CH₃ (MOM) group, a —O—CH₂—O—CH₂—CH₂—O—CH₃ (MEM) group, a —OSO₂-alkyl group or a —OSi(CH₃)₂tBu;
D represents an oxygen atom, NR₃, CR′R″ or a sulfur atom;
X represents an oxygen atom, a sulfur atom or a radical —NR₄—;
Y represents an oxygen atom, a sulfur atom or a radical —NR₄—;
R₃ represents a hydrogen, an alkyl group, a carboxylate group, an acyl group, a carboxamide group or a SO₂-alkyl group;
R′ and R″, identical or different, represent a hydrogen atom or an alkyl radical;
R₄, identical or different, is selected from a group consisting of a hydrogen atom, an alkyl group having from 1 to 10 carbon atoms, an aryl and an aralkyl;
“linker” represents (CH₂)ₙ, wherein n represents an integer between 1 and 10 inclusive, optionally interrupted by an heteroatom (preferably N, O, S and P) or a carbonyl group, or an aryidialkyl (preferably xylenyl) group;
A represents a group selected from:

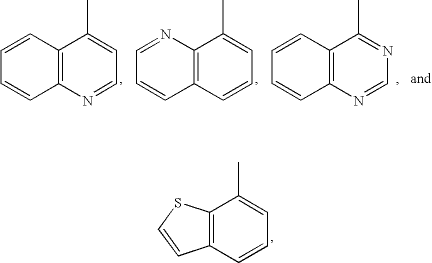

optionnally A is substituted,
its tautomers, optical and geometrical isomers, racemates, salts, hydrates and mixtures thereof.

The above compounds may have one or more asymmetric centers and it is intended that stereoisomers (optical isomers), as separated, pure or partially purified stereoisomers or racemic mixtures thereof are included in the scope of the invention.

As will be further disclosed in this application, compounds of formula (I) above are potent, brain penetrant molecules active at inhibiting Rac1 and APP processing, lowering Aβ production in vitro and in vivo.

Within the context of the present application, the terms alkyl and alkoxy denote linear or branched saturated groups containing from 1 to 10 carbon atoms. An alkoxy group denotes an —O-alkyl group.

The alkyl groups may be linear or branched. Examples of alkyl groups having from 1 to 10 carbon atoms inclusive are methyl, ethyl, propyl, isopropyl, t-butyl, n-butyl, pentyl, hexyl, heptyl, octyl, nonyl, decyl, 2-ethylhexyl, 2-methylbutyl, 2-methylpentyl, 1-methylhexyl, 3-methylheptyl and the other isomeric forms thereof. Preferably, the alkyl groups have from 1 to 6 carbon atoms.

The alkenyl groups may be linear or branched. Examples of alkenyl containing from 3 to 6 carbon atoms are 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl and the isomeric forms thereof.

The term aryl includes any aromatic group comprising preferably from 5 to 14 carbon atoms, preferably from 6 to 14 carbon atoms, optionally interrupted by one or several heteroatoms selected from N, O, S or P. Most preferred aryl groups are mono- or bi-cyclic and comprises from 6 to 14 carbon atoms, such as phenyl, α-naphtyl, β-naphtyl, antracenyl, or fluorenyl group.

The term aralkyl group generally stands for an aryl group attached to an alkyl group as defined above, such as benzyl or phenethyl.

The term carboxylate group generally stands for a group presenting a —COO—R radical, wherein R represents a hydrogen atom, an aryl group, or preferably an alkyl radical. In this respect, R3 represents preferably a tert-butyl-carboxylate group.

The term acyl group generally stands for a —COR group, wherein R represents an aryl group, or preferably an alkyl radical. In this respect, R3 represents preferably an acetyl, a pivaloyl, or a benzoyl group.

The term carboxamide group generally stands for a —CONR′R″ group, wherein R′ and R″, identical or different, are as defined above. In this respect, R3 represents preferably an N,N-diethyl- or N,N-diisopropyl-carboxamide group or a N-tert-butyl- or N-methyl-carboxamide group.

According to a particular embodiment, A is substituted with at least one substituent, which may be selected from the group consisting of: a hydrogen atom, a halogen atom (preferably F, Cl, or Br), a hydroxyl group, a (C₁-C₁₀)alkyl group, an alkenyl group, an (C₁-C₁₀)alkanoyl group, a (C₁-C₁₀)alkoxy group, an (C₁-C₁₀)alkoxycarbonyl (or carboxylate) group, an aryl group, an aralkyl group, an arylcarbonyl group, a mono- or poly-cyclic hydrocarbon group, a —NHCO(C₁-C₆)alkyl group, —NO₂, —CN, a —NR₅R₆ group or a trifluoro(C₁-C₆)alkyl group, R₅ and R₆, independently from each other, are selected from the group consisting of a hydrogen atom, an alkyl group having from 1 to 10 carbon atoms, an aryl and an aralkyl.

An alkanoyl group is a —CO-alkyl group, the alkyl group being as defined above.

The term arylcarbonyl group generally stands for an aryl group attached to a carbonyl group, the aryl group being as defined above.

The term alkoxycarbonyl group generally stands for an alkoxy group attached to a carbonyl group, the alkoxy group being as defined above.

The term mono- or poly-cyclic hydrocarbon group is understood to refer to hydrocarbon cyclic group having from 1 to 20 carbon atoms, optionally interrupted with one or more heteroatoms selected in the group N, O, S and P. Among such mono- or poly-cyclic hydrocarbon groups, cyclopentyl, cyclohexyl, cycloheptyl, 1- or 2-adamantyl groups, pyran, piperidine, pyrrolidine, morpholine, dioxan, tetrahydrothiophene, and tetrahydrofuran can be cited. The mono- or poly-cyclic hydrocarbon group may form with the phenyl group it is attached an aryl group, such as a α-naphtyl, β-naphtyl, or antracenyl group.

Where the linker represents (CH₂)ₙ, interrupted by an heteroatom, the heteroatom is more preferably an oxygen atom. In this case, the linker is advantageously a —CH₂CH₂OCH₂CH₂— group. Where the linker represents (CH₂)ₙ interrupted by a carbonyl group, said linker may represent a —(C═O)CH₂CH₂CH₂CH₂— group (preferably when X is —NR4—). The groups identified above may be optionally substituted. In particular, the alkyl, alkenyl, aryl, aralkyl, and the mono- or poly-cyclic hydrocarbon group may be optionally substituted with one or more groups selected from hydroxyl group, halogen atom, cyano group, nitro group, ester (—COO(C₁-C₆)alkyl group), —OCO(C₁-C₆)alkyl group, amide (—NHCO(C₁-C₆)alkyl or —CONH(C₁-C₆)alkyl group), (C₁-C₁₀)alkyl radical, (C₁-C₁₀)alkoxy radical, mono- or poly-cyclic hydrocarbon group, C═O group, a —NR₅R₆ group or a trifluoro(C₁-C₆)alkyl group, R₅ and R₆ being as defined above.

The trifluoro(C₁-C₆)alkyl group is preferably the trifluoromethyl group.

According to preferred embodiments, the compounds according to the invention correspond to general formula (I) wherein:
X is sulfur, —NH— or oxygen; and/or
Y is oxygen; and/or
“linker” represents (CH₂)ₙ, wherein n is from 2 to 9, preferably 4 to 7, inclusive, or the meta, ortho or para-xylenyl groups, —CH₂CH₂OCH₂CH₂— and —(C═O)CH₂CH₂CH₂CH₂—); and/or
R₁ is

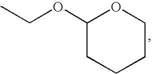

—CH₂N(Et₂) and —CH₂pyrrolidine,

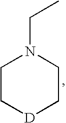

wherein D is oxygen, sulfur, —CH₂— or NR₃, wherein R₃ preferably represents H or an alkyl group (said alkyl is more specifically a methyl radical), and —CH₂—B, wherein B is a —O—CH₂—O—CH₃ group or —OSO₂-alkyl group (wherein alkyl is preferably methyl) or halogen (preferably chlorine of fluorine); and/or
R₂ is a hydrogen atom; and/or
A is a substituted group as defined above.

In a particular embodiment, when A is a substituted group as defined above, at least one of the substituents is a halogen atom, more preferably chlorine or fluorine.

A particular preferred group of compounds according to the present invention, are the compounds of formula (I) wherein at least one of the substituents, and more preferably all the substituents, of A represents a hydrogen atom, a methyl group, a propyl group, an ethoxy group, an halogen atom, preferably chlorine or fluorine, or the CF₃ group.

Most preferred compounds for use in the present invention correspond to general formula (I) wherein
X is sulfur; and/or
Y is oxygen; and/or
“linker” represents (CH₂)ₙ, wherein n is from 4 to 7, inclusive; and/or
R₁ is

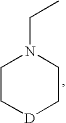

wherein D is oxygen, and/or
R₂ is a hydrogen atom; and/or
A is a group of formula

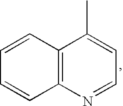

optionnally substituted, most preferably by a trifluoro(C₁-C₆)alkyl group, particularly the CF₃ group.

When the compounds according to the invention are in the forms of salts, they are preferably pharmaceutically acceptable salts. Such salts include pharmaceutically acceptable acid addition salts, pharmaceutically acceptable base addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like. Further examples of pharmaceutically acceptable inorganic or organic acid addition salts include the pharmaceutically acceptable salts listed in J. Pharm. Sci. 1977, 66, 2, which is incorporated herein by reference. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like.

The pharmaceutically acceptable salts are prepared by reacting the compound of formula I with 1 to 4 equivalents of a base such as sodium hydroxide, sodium methoxide, sodium hydride, potassium t-butoxide, calcium hydroxide, magnesium hydroxide and the like, in solvents like ether, THF, methanol, t-butanol, dioxane, isopropanol, ethanol, etc. Mixture of solvents may be used. Organic bases like lysine, arginine, diethanolamine, choline, guanidine and their derivatives etc. may also be used. Alternatively, acid addition salts wherever applicable are prepared by treatment with acids such as hydrochloric acid, hydrobromic acid, nitric acid, sulfuric acid, phosphoric acid, p-toluenesulphonic acid, methanesulfonic acid, fonic acid, acetic acid, citric acid, maleic acid, salicylic acid, hydroxynaphthoic acid, ascorbic acid, palmitic acid, succinic acid, benzoic acid, benzenesulfonic acid, tartaric acid and the like in solvents like ethyl acetate, ether, alcohols, acetone, THF, dioxane, etc. Mixture of solvents may also be used.

Specific examples of compounds of formula (I) which fall within the scope of the present invention include the following compounds:
2-(Tetrahydro-pyran-2-yloxymethyl)-5-[5-(7-trifluoromethyl-quinolin-4-yloxy)-pentyloxy]-pyran-4-one (1)
5-[5-(6-Fluoro-2-methyl-quinolin-4-yloxy)-pentyloxy]-2-(tetrahydro-pyran-2-yloxymethyl)-4H-pyran-4-one (2)
5-[5-(6-Fluoro-2-trifluoromethyl-quinolin-4-yloxy)-pentyloxy]-2-(tetrahydro-pyran-2-yloxymethyl)-4H-pyran-4-one (3)
5-[5-(7-Propyl-quinolin-8-yloxy)-pentyloxy]-2-(tetrahydro-pyran-2-yloxymethyl)-4H-pyran-4-one (4)
5-[5-(Benzo[b]thiophen-7-yloxy)-pentyloxy]-2-(tetrahydro-pyran-2-yloxymethyl)-4H-pyran-4-one (5)
2-(Tetrahydro-pyran-2-yloxymethyl)-5-[5-(7-trifluoromethyl-quinolin-4-ylsulfanyl)-pentyloxy]-4H-pyran-4-one (6)
2-(Tetrahydro-pyran-2-yloxymethyl)-5-[4-(7-trifluoromethyl-quinolin-4-ylsulfanyl)-butoxy]-4H-pyran-4-one (7)
2-(Tetrahydro-pyran-2-yloxymethyl)-5-[6-(7-trifluoromethyl-quinolin-4-ylsulfanyl)-hexyloxy]-4H-pyran-4-one (8)
2-Hydroxymethyl-5-[5-(7-trifluoromethyl-quinolin-4-ylsulfanyl)-pentyloxy]-4H-pyran-4-one hydrochloride salt (9)
2-Hydroxymethyl-5-[5-(7-trifluoromethyl-quinolin-4-ylsulfanyl)-pentyloxy]-4H-pyran-4-one (10)
2-Methoxymethoxymethyl-5-[5-(7-trifluoromethyl-quinolin-4-ylsulfanyl)-pentyloxy]-4H-pyran-4-one (11)
2-Chloromethyl-5-[5-(7-trifluoromethyl-quinolin-4-ylsulfanyl)-pentyloxy]-4H-pyran-4-one (12)
2-(4-Methyl-piperazin-1-ylmethyl)-5-[5-(7-trifluoromethyl-quinolin-4-ylsulfanyl)-pentyloxy]-4H-pyran-4-one (13)
2-Morpholin-4-ylmethyl-5-[5-(7-trifluoromethyl-quinolin-4-ylsulfanyl)-pentyloxy]-4H-pyran-4-one (14)
5-(5-(7-(Trifluoromethyl)quinolin-4-ylthio)pentyloxy)-2-(fluoromethyl)-4H-pyran-4-one (15)
5-(5-(7-(Trifluoromethyl)quinolin-4-ylthio)pentyloxy)-2-((piperidin-1-yl)methyl)-4H-pyran-4-one (16)
5-(5-(7-(Trifluoromethyl)quinolin-4-ylthio)pentyloxy)-2-(thiomorpholino-methyl)-4H-pyran-4-one (17)
2-((Diethylamino)methyl)-5-(5-(7-(trifluoromethyl)quinolin-4-ylthio)pentyloxy)-4H-pyran-4-one (18)
4-[5-(6-Morpholin-4-ylmethyl-4-oxo-4H-pyran-3-yloxy)-pentyloxy]-7-trifluoromethyl-quinoline-3-carboxylic acid ethyl ester (19)
5-(5-(8-(Trifluoromethyl)quinolin-4-yloxy)pentyloxy)-2-((4-methylpiperazin-1-yl)methyl)-4H-pyran-4-one (20)
5-(5-(8-(Trifluoromethyl)quinolin-4-yloxy)pentyloxy)-2-(morpholinomethyl)-4H-pyran-4-one (21)
5-(5-(7-(Trifluoromethyl)quinolin-4-yloxy)pentyloxy)-2-(morpholinomethyl)-4H-pyran-4-one (22)
5-(5-(7-(Trifluoromethyl)quinolin-4-yloxy)pentyloxy)-2-((4-methylpiperazin-1-yl)methyl)-4H-pyran-4-one (23)
5-(5-(6-(Trifluoromethyl)quinolin-4-yloxy)pentyloxy)-2-(morpholinomethyl)-4H-pyran-4-one (24)
4-[5-(6-(4-Methyl-piperazin-1-ylmethyl)-4-oxo-4H-pyran-3-yloxy)-pentyloxy]-7-trifluoromethyl-quinoline-3-carboxylic acid ethyl ester (25)
4-[5-(6-(4-Methyl-piperazin-1-ylmethyl)-4-oxo-4H-pyran-3-yloxy)-pentyloxy]-7-trifluoromethyl-quinoline-3-carboxylic acid ethyl ester (26)
5-((4-((7-(Trifluoromethyl)quinolin-4-ylthio)methyl)phenyl)methoxy)-2-(morpholinomethyl)-4H-pyran-4-one (27)
5-((2-((7-(Trifluoromethyl)quinolin-4-ylthio)methyl)phenyl)methoxy)-2-(morpholinomethyl)-4H-pyran-4-one (28)
5-(5-(7-(Trifluoromethyl)quinolin-4-ylthio)pentyloxy)-2-((4-acetylpiperazin-1-yl)methyl)-4H-pyran-4-one (29)
4-((5-(5-(7-(Trifluoromethyl)quinolin-4-ylthio)pentyloxy)-4-oxo-4H-pyran-2-yl)methyl)-N,N-diethylpiperazine-1-carboxamide (30)
5-(5-(7-(Trifluoromethyl)quinolin-4-ylthio)pentyloxy)-2-((4-(pivaloyl)piperazin-1-yl)methyl)-4H-pyran-4-one (31)
4-((5-(5-(7-(Trifluoromethyl)quinolin-4-ylthio)pentyloxy)-4-oxo-4H-pyran-2-yl)methyl)-N,N-di-isopropylpiperazine-1-carboxamide (32)
5-(5-(7-(Trifluoromethyl)quinolin-4-ylthio)pentyloxy)-2-((4-methylsulfonylpiperazin-1-yl)methyl)-4H-pyran-4-one (33)
4-((5-(5-(7-(Trifluoromethyl)quinolin-4-ylthio)pentyloxy)-4-oxo-4H-pyran-2-yl)methyl)-N-tert-butylpiperazine-1-carboxamide (34)
4-((5-(5-(7-(Trifluoromethyl)quinolin-4-ylthio)pentyloxy)-4-oxo-4H-pyran-2-yl)methyl)-N-methylpiperazine-1-carboxamide (35)
5-(6-(Morpholinomethyl)-4-oxo-4H-pyran-3-yloxy)-N-(7-(trifluoromethyl)quinolin-4-yl)pentanamide (36)
5-(2-(2-(7-(Trifluoromethyl)quinolin-4-ylthio)ethoxy)ethoxy)-2-(morpholinomethyl)-4H-pyran-4-one (37)
5-(5-(7-(Trifluoromethyl)quinolin-4-ylthio)pentyloxy)-2-(morpholinomethyl)-4H-pyran-4-one dihydrochloride (38)
5-((3-((7-(Trifluoromethyl)quinolin-4-ylthio)methyl)phenyl)methoxy)-2-(morpholinomethyl)-4H-pyran-4-one dihydrochloride (39)
tert-Butyl 4-((5-(5-(7-(trifluoromethyl)quinolin-4-ylthio)pentyloxy)-4-oxo-4H-pyran-2-yl)methyl)piperazine-1-carboxylate (40)
tert-Butyl 4-((5-(5-(7-chloroquinolin-4-yloxy)pentyloxy)-4-oxo-4H-pyran-2-yl)methyl)piperazine-1-carboxylate (41)
5-(5-(7-(Trifluoromethyl)quinolin-4-ylthio)pentyloxy)-2-((4-methylpiperazin-1-yl)methyl)-4H-pyran-4-one trihydrochloride (42)
5-(5-(7-(Trifluoromethyl)quinolin-4-ylthio)pentyloxy)-2-((piperazin-1-yl)methyl)-4H-pyran-4-one trihydrochloride (43)
5-(5-(7-Chloroquinolin-4-yloxy)pentyloxy)-2-((piperazin-1-yl)methyl)-4H-pyran-4-one (44)
5-(2-(7-(Trifluoromethyl)quinolin-4-ylthio)ethoxy)-2-((tetrahydro-2H-pyran-2-yloxy)methyl)-4H-pyran-4-one (45)
5-(8-(7-(Trifluoromethyl)quinolin-4-ylthio)octyloxy)-2-((tetrahydro-2H-pyran-2-yloxy)methyl)-4H-pyran-4-one (46)
5-(7-(7-(Trifluoromethyl)quinolin-4-ylthio)heptyloxy)-2-((tetrahydro-2H-pyran-2-yloxy)methyl)-4H-pyran-4-one (47)
5-(2-(7-(Trifluoromethyl)quinolin-4-yloxy)ethoxy)-2-(morpholinomethyl)-4H-pyran-4-one (48).

A particularly preferred compound is 5-(5-(7-(Trifluoromethyl)quinolin-4-ylthio)pentyloxy)-2-(morpholinomethyl)-4H-pyran-4-one dihydrochloride (38), in the form of a free base or any pharmaceutically acceptable salt thereof.

The compounds according to the present invention may be prepared by various methods known to those skilled in the art. Such methods are disclosed in WO2004/076445, which is incorporated therein by reference. It should be understood that other ways of producing these compounds may be designed by the skilled person, based on common general knowledge and following guidance contained in this application.

A particular object of this invention relates to a method of treating an amyloid beta peptide-related disorder in a mammalian subject, comprising administering to a subject in need thereof an amount of a Rac1 inhibitor of formula (I) above effective at reducing APP processing in said subject.

A further particular object of this invention relates to a method of treating an amyloïd beta peptide-related disorder in a mammalian subject, comprising administering to a subject in need thereof an amount of a Rac1B and/or Rac2 inhibitor, e.g., of formula (I) above, effective at reducing APP processing in said subject.

A further aspect of this invention is a method of inhibiting the generation of an amyloïd beta peptide in a mammalian subject, comprising administering to a subject in need thereof an amount of a Rac1 inhibitor of formula (I) effective at reducing APP processing in said subject.

A further aspect of this invention is a method of inhibiting the generation of an amyloïd beta peptide in a mammalian subject without substantially altering the Notch cleavage or BACE activity, comprising administering to a subject in need thereof an effective amount of a Rac1 inhibitor of formula (I) above.

A particular object of this invention relates to a method of treating an amyloïd beta peptide-related disorder in a mammalian subject, comprising administering to a subject in need thereof an amount of a compound of formula (I) above effective at reducing APP processing in said subject.

A further aspect of this invention is a method of inhibiting the generation of an amyloïd beta peptide in a mammalian subject, comprising administering to a subject in need thereof an amount of a compound of formula (I) effective at reducing APP processing in said subject.

A further aspect of this invention is a method of inhibiting the generation of an amyloïd beta peptide in a mammalian subject without substantially altering the Notch cleavage or BACE activity, comprising administering to a subject in need thereof an effective amount of a compound of formula (I) above.

According to a particular embodiment, the compounds used in the present invention inhibit Rac1 and Rac1 B.

According to an other particular embodiment, the compounds used in the present invention inhibit Rac1 and Rac2.

According to a particular embodiment, the compounds used in the present invention inhibit Rac1, Rac1 B and Rac2.

According to an other particular embodiment, the compounds used in the present invention inhibit Rac3, preferably Rac1 and Rac3, particularly Rac1, Rac1 B and Rac3 or Rac1, Rac1 B, Rac2 and Rac3.

Preferred compounds for use according to the invention include any sub-group as defined above, as well as each of the specific compounds listed above.

In an other particular embodiment, the Rac1 inhibitor is compound NSC23766 (compound 49) or a derivative thereof. The structure of compound 49 is represented below:

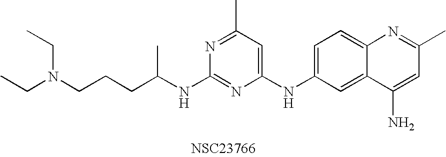

In a further particular embodiment, the Rac1 inhibitor is a compound obtained, selected, identified, optimised or produced by a method of this invention, as disclosed below.

For use in the present invention, the compounds may be in the form of a pharmaceutical composition comprising at least one of said compounds and a pharmaceutically acceptable vehicle or support. The compounds may be formulated in various forms, including solid and liquid forms, such as capsules, tablets, gel, solution, syrup, suspension, powder, aerosol, oitment, etc.

Such pharmaceutical compositions of this invention may contain physiologically acceptable diluents, fillers, lubricants, excipients, solvents, binders, stabilizers, and the like. Diluents that may be used in the compositions include but are not limited to dicalcium phosphate, calcium sulphate, lactose, cellulose, kaolin, mannitol, sodium chloride, dry starch, powdered sugar and for prolonged release tablet-hydroxy propyl methyl cellulose (HPMC). The binders that may be used in the compositions include but are not limited to starch, gelatin and fillers such as sucrose, glucose, dextrose and lactose.

Natural and synthetic gums that may be used in the compositions include but are not limited to sodium alginate, ghatti gum, carboxymethyl cellulose, methyl cellulose, polyvinyl pyrrolidone and veegum. Excipients that may be used in the compositions include but are not limited to microcrystalline cellulose, calcium sulfate, dicalcium phosphate, starch, magnesium stearate, lactose, and sucrose. Stabilizers that may be used include but are not limited to polysaccharides such as acacia, agar, alginic acid, guar gum and tragacanth, amphotsics such as gelatin and synthetic and semi-synthetic polymers such as carbomer resins, cellulose ethers and carboxymethyl chitin.

Solvents that may be used include but are not limited to Ringers solution, water, distilled water, dimethyl sulfoxide to 50% in water, propylene glycol (neat or in water), phosphate buffered saline, balanced salt solution, glycol and other conventional fluids.

The dosages and dosage regimen in which the compounds are administered will vary according to the dosage form, mode of administration, the condition being treated and particulars of the patient being treated. Accordingly, optimal therapeutic concentrations will be best determined at the time and place through routine experimentation.

The compounds according to the invention can also be used enterally. Orally, the compounds according to the invention are suitable administered at the rate of 10 μg to 300 mg per day per kg of body weight. The required dose can be administered in one or more portions. For oral administration, suitable forms are, for example, capsules, tablets, gel, aerosols, pills, dragees, syrups, suspensions, emulsions, solutions, powders and granules; a preferred method of administration consists in using a suitable form containing from 1 mg to about 500 mg of active substance.

The compounds according to the invention can also be administered parenterally in the form of solutions or suspensions for intravenous, subcutaneous or intramuscular perfusions or injections. In that case, the compounds according to the invention are generally administered at the rate of about 10 μg to 10 mg per day per kg of body weight; a preferred method of administration consists of using solutions or suspensions containing approximately from 0.01 mg to 1 mg of active substance per ml.

The compounds according to the invention can also be administered in the eye in the form of solutions or suspensions for intravitreous or retro-orbitary injections. In that case, the compounds according to the invention are generally administered at the rate of about 10 μg to 10 mg per day per kg of body weight; a preferred method of administration consists of using solutions, suspensions or gel containing approximately from 0.01 mg to 1 mg of active substance per ml.

The compounds can be used in a substantially similar manner to other known agents for treating CNS disorders. The dose to be administered, whether a single dose, multiple dose, or a daily dose, will vary with the particular compound employed because of the varying potency of the compound, the chosen route of administration, the size of the recipient, the type of disease and the nature of the patient's condition. The dosage to be administered is not subject to definite bounds, but it will usually be an effective amount, or the equivalent on a molar basis of the pharmacologically active free form produced from a dosage formulation upon the metabolic release of the active drug to achieve its desired pharmacological and physiological effects. An physician or a doctor skilled in the art of CNS disorder treatment will be able to ascertain, without undue experimentation, appropriate protocols for the effective administration of the compounds of this invention.

The compounds may be administered according to various routes, typically by oral route or by injection, such as local or systemic injection(s). Oral, intraveinous, intraperitoneal or sub-cutaneous administration are preferred, although other administration routes may be used as well, such as intramuscular, intradermic, etc. Furthermore, repeated injections may be performed, if appropriate.

A particular object of this invention relates to a method of treating Alzheimer's disease in a mammalian subject, comprising administering to a subject in need thereof an amount of a compound of formula (I) above effective at reducing APP processing in said subject.

A further aspect of this invention is a method of inhibiting the generation of an amyloïd beta peptide in a mammalian subject having Alzheimer's disease, comprising administering to said subject an amount of a compound of formula (I) effective at reducing APP processing in said subject.

A further aspect of this invention is a method of inhibiting the generation of an amyloïd beta peptide in a mammalian subject having Alzheimer's disease without substantially altering the Notch cleavage or BACE activity, comprising administering to said subject an effective amount of a compound of formula (I) above.

In a most preferred embodiment, the compound is 5-(5-(7-(Trifluoromethyl)quinolin-4-ylthio)pentyloxy)-2-(morpholinomethyl)-4H-pyran-4-one dihydrochloride (38), in the form of a free base or any pharmaceutically acceptable salt thereof.

A further object of this invention is the use of a Rac1 inhibitor for the preparation of a pharmaceutical composition for treating Alzheimer's disease.

### Drug Identification

The invention implicates, for the first time, Rac-1 in the modulation of APP processing and Aβ generation. Accordingly, the invention shows that Rac1 represents a valuable target for therapeutic intervention in any disease associated with Aβ generation, and for the screening of drugs to be used in the treatment of such diseases.

In this respect, a particular object of this invention relates to methods of producing, identifying, selecting or optimising candidate compounds for use in the treatment of amyloïd beta peptide-related disorders, the method comprising determining whether a test compound inhibits Rac1, Rac1 inhibition being an indication that the test compound is a candidate compound for use in the treatment of amyloïd beta peptide-related disorders. Rac1 inhibition may be assessed in vitro, ex vivo or in vivo, according to various biological assays which are known per se in the art.

In a particular embodiment, the method comprises contacting the test compound and Rac1 (or a fragment thereof) and determining whether the compound binds Rac1 or the fragment thereof.

In an other particular embodiment, the method comprises contacting the test compound and Rac1 and determining whether the compound inhibits Rac1-dependent cytoskeleton rearrangements.

In a particular embodiment, Rac1 inhibition is assessed using the effector PAK1 pull-down assay, as disclosed in the examples.

More preferably, the compounds are further assessed for their activity towards other targets, particularly the Notch processing pathway (e.g., Notch cleavage), BACE, or other small GTP-binding proteins (e.g., Cdc42 and/or RhoA). Most preferred compounds are those which substantially do not alter Notch cleavage and/or do not substantially directly inhibit BACE, and/or do not substantially directly inhibit Cdc42 and/or RhoA.

The assays may be conducted in any suitable device, and various test compounds may be assayed in parallel, or in mixtures.

Further aspects and advantages of this invention will be disclosed in the following examples, which should be regarded as illustrative and not limiting the scope of this application.

### EXAMPLES

### Example 1

### Experimental Procedures

### Materials and Compounds

The synthesis of compound 38 is disclosed in Example 2. All cell culture reagents were from Invitrogen unless otherwise noted. NSC23766, DAPT, BACE inhibitors, BACE and γ-secretase fluorogenic substrates were obtained from Calbiochem.

Cell Culture and treatments—Stably transfected HEK293 cells overexpressing human swAPP harboring the “Swedish” mutation (Chevallier, N., Jiracek, J., Vincent, B., Baur, C. P., Spillantini, M. G., Goedert, M., Dive, V., and Checker, F. (1997) Br. J. Pharmacol. 121, 556-562) (swAPP-HEK293 cells) were maintained in Modified Eagle's medium+Earle's salt supplemented with 10% fetal bovine serum (FBS), 2 mM L-glutamine (Sigma), 1× Non-Essential Amino Acids and antibiotics. NIH3T3 cells (Lgc PromoChem) were grown in High Glucose DMEM+Glutamax supplemented with 10% New Born Serum and antibiotics. Human glioblastoma astrocytoma U87MG (ATCC # HTB-14) were grown at 37° C. in DMEM containing 1 mM glutamine, 10% FBS and antibiotics. SH-SY5Y cells (ATCC # CRL-2266) were maintained in Modified Eagle's medium/F12K (1:1, v/v) supplemented with 10% FBS, 2 mM L-glutamine, 1× Non-Essential Amino Acids, 1× Sodium Pyruvate and antibiotics. Hela cells (ATCC # CCL 2) were grown in Modified Eagle's medium supplemented with 10% FBS, 2 mM L-glutamine and antibiotics. Cells were treated 48 hours after plating in 10 cm plates with various concentrations of the indicated molecules, or DMSO as the vehicle for 16 hours. To do so, medium was replaced with 5 ml of new medium in which treatments were performed. Total DMSO dilution was 1/1000 in all cases. Cells were allowed to secrete in 5 ml medium for 7 hours in the presence of 1 μM phosphoramidon.

### Endogenous Rac GTPase Activation Assay

U87-MG cells were grown in a 150-mm-diameter dish until they reached 80% confluency. The cells were then treated with the test compounds or the solvent only. Cells were then lyzed in a buffer containing 0.5% triton, 10 mM Tris pH7.5, mM KCl, 120 mM NaCl and 1.8 mM CaCl₂. Lysates were clarified, the protein concentrations were normalized, and the GTP-bound Rac1 in the lysates were measured using the Rac Activation Assay Biochem kit (Cytoskeleton) as per manufacturer's recommendations.

### Transient Expression Reporter Assays

Transcriptional activation of luciferase gene expression constructs was performed as described previously (Whitehead, I. P., Lambert, Q. T., Glaven, J. A., Abe, K., Rossman, K. L., Mahon, G. M., Trzaskos, J. M., Kay, R., Campbell, S. L., and Der, C. J. (1999) Mol. Cell. Biol. 19, 7759-7770). Briefly, 250,000 NIH3T3 cells/well were seeded in 6-well plates and were co-transfected 24 h later with plasmids prK5-RacV12 and reporter constructs using LipofectAMINE Plus (Invitrogen). Compound of interest was added after the incubation with LipofectAmine. 24 h after transfection, cells were starved for an additional 24 h with Dulbecco's modified Eagle medium supplemented with 0.5% FBS together with the appropriate doses of test compounds or the solvent only. Analyses of the cell lysates of the transiently transfected NIH3T3 cells were performed using the Luciferase Assay System (Promega) and Fluoroscan Ascent FL plate reader (Thermo LabSystems). All assays were performed in duplicate, and results shown represent the mean (±standard error mean [SEM]) of four independent experiments for each reporter gene. We did not use internal standard in the transfections, since all 3 promoters tested responded to active Rac overexpression to varying extents. However, consistent and reproducible data were obtained in different assays performed using several plasmid preparations, and we monitored protein concentration for yield in the cell extracts as well as expression of the tagged, exogenous protein by Western blotting.

The reporter constructs 5×Gal-4-Luc plus Gal-4-c-Jun, HIV-Luc bearing NF-KB binding sites, and cyclin D1-Luc (Albanese, C., J. Johnson, G. Watanabe, N. Eklund, D. Vu, A. Arnold, and Pestell, R. G. (1995) J. Biol. Chem. 270, 23589-23597) were described previously and are a kind gift of Professor Channing J. Der (University of North Carolina, Chapel Hills, N.C.). The expression plasmid prK5-RacV12 was described previously and is a kind gift of Professor Alan Hall (University College London, UK).

### Western Blot Analyses

swAPP-HEK293 cells were scraped and lysed in CelLytic-M (Sigma). Protein concentrations were determined by the Bradford procedure. Equal quantity of proteins were separated on a 10% SDS-PAGE gel and transferred to Hybond-C (Amersham Biosciences) membranes. After transfer, membranes were blocked with 5% nonfat milk and incubated overnight with the primary antibody anti-APP antibody at 1/1000 (Serotec), allowing the detection of both APP and C83-C99 CTFs under specific separation and exposure conditions. For sAPPα detection, cells were allowed to secrete for 7 h. Media were collected, centrifuged and then equal amount of secretate were loaded on 10% SDS-PAGE and Western blotted with 6E10 monoclonal antibody (1/1000). Immunological complexes were revealed with an anti-mouse peroxidase (Jakson Laboratories, 1/5000) antibody followed by ECL enhanced chemiluminescence (Amersham Biosciences).

### NotchΔE Transfection and Notch-1 Cleavage Assays in Hela Cells

Hela cells in 10 cm plates were transiently transfected with the expression vector pSC2+ΔE3MV-6MT which allows overexpression of the truncated Notch-1 lacking most of the Notch extracellular domain with a C-terminal hemagglutinin tag, NotchΔE), which is the substrate of γ-secretase (Kopan, R., Schroeter, E. H., Weintraub, H. And Nye, J. S. (1996) Proc. Natl. Acad. Sci. USA 93, 1683-1688). One day post-transfection, cultures were preincubated with compound 38 or the γ-secretase inhibitor DAPT for 18 h at the indicated concentrations, then CelLytic-M lysates were processed for detection of the Notch Intracellar Domain (NICD) by Western blotting using anti-myc antibody (Santa Cruz Biotechnology) at 1/1000.

### In Vivo Delivery of Inhibitors

Compound 38 or vehicle (DMSO) was injected in Male Hartley albino guinea-pigs, weighing 250-270 g at delivery, obtained from Charles River Laboratories (L'Arbresle, France), once a day for 15 consecutive days and by the i.p. route. One hour after the final administration, the guinea-pigs were killed and brains were immediately extracted and immersed in an oxygenated (95% O₂/5% CO₂) physiological saline bath placed on ice (1-2° C.) and superficial vessels were removed. The whole brains were dissected to provide left and right cortices, which were weighted, snap frozen in liquid nitrogen, and stored at −80° C. separately. The maximum time between sacrifice and snap freezing was less than 15 minutes.

### Measurements of Aβ 40 and Aβ 42

Stably transfected swAPP-HEK293 cells were incubated for 7 hours in the presence of phosphoramidon (1 μM) (Sigma). Media and cell lysates were collected as above, centrifuged, normalized to total protein and assayed for Aβ 40 and Aβ 42 by sandwich ELISA according to the manufacturer's instructions (Biosource International). For Aβ 42 detection, samples were concentrated on YM3 Microcon columns (Millipore). For in vivo samples, the protocol ensured a final concentration of guanidine inferior to 0.1 M, as recommended by the manufacturer and ELISA standards included guanidine. Right cortices were homogenized for 3 h at room temperature in 5M Guanidine-Hcl, 50 mM Tris-Hcl, pH8 with a protease inhibitor mixture (Roche Diagnostics). Tissue homogenates were diluted 1:1 (v/v) in BSAT-DPBS buffer (Dulbecco's phosphate buffered saline with 5% BSA and 0.03% Tween-20), pH 7.4, and were centrifuged at 20,000 g for 20 min at 4° C. Supernatants were diluted 1:1 (v/v) in ELISA kit sample buffer, normalized to total protein and assayed for Aβ 40 and Aβ 42 by sandwich ELISA according to the manufacturer's instructions. For Aβ 42 detection, samples were concentrated on YM3 Microcon columns (Millipore).

### BACE Assay

The human BACE1 cDNA was generated by RT-PCR from human brain mRNA samples (Biocat, Germany) and cloned into pcDNA3 expression vector. Subsequently, a HEK293 cell line stably expressing BACE1 was generated and used as a source of BACE1. An in vitro assay was developed based on previous studies (Ermolieff, J., Loy, J. A., Koelsch, G., and Tang, J. (2000) Biochemistry 39, 12450-12456) using a quenched fluorogenic substrate containing the Swedish mutation MCA-SEVNLDAEFK(DNP)-CONH₂ (Substrate V, Calbiochem). Proteins were extracted in 20 mM MES/1% Triton X100 plus protease inhibitor cocktail by incubation on ice for 30 minutes. The assay was carried out in black 96 well plates (ATGC) in a volume of 200 μl reaction buffer (25 mM MES/25 mM Sodium Acetate/25 mM Tris, pH 4.4), containing 25 μl of the preparation plus 15 μM peptide Substrate V. Excitation was performed at 320 nm and the reaction kinetics were monitored by measuring the fluorescence emission at 420 nm on a Fluoroscan Ascent FL plate reader (Thermo LabSystems). Controls included purified recombinant human BACE501 protein (R&D Systems) diluted at 1 μg/well in 200 μl of 0.1 M Na Acetate buffer (pH 4.4), the BACE substrate analog inhibitor III (H-Glu-Val-Asn-Statine-Val-Ala-Glu-Phe-NH2, Calbiochem), or substrate alone, and background fluorescence was subtracted to recorded BACE activitiy. Final DMSO concentration was 1% (v/v) and did not affect the fluorescence or BACE activity.

### γ-Secretase Assay

We implemented a an assay allowing de novo Aβ generation in vitro, using cell membranes as the source of γ-secretase. Preparation of solubilized γ-secretase fractions was performed essentially as described previously (Li, Y. M., Lai, M. T., Xu, M., Huang, Q., DiMuzio-Mower, J., Sardana, M. K., Shi, X. P., Yin, K. C., Shafer, J. A., and Gardell, S. J. (2000) Proc. Natl. Acad. Sci. U.S.A. 97, 6138-6143) with the above modifications. All incubations were performed in the presence of Complete Protease Inhibitor Cocktail. Confluent plates swAPP-HEK293 cells were lysed in 1 ml of ice-cold CelLytic-M (Sigma) and incubated 15 minutes at 4° C. on a shaker. Cell debris and nuclei were removed by centrifugation at 1000×g for 15 min at 4° C. For membrane isolation, the supernatant solutions were centrifuged at 20,000×g for 1 hour at 4° C. After centrifugation, the ensuing pellets were resuspended in 100 μl activity buffer (150 mM Na Citrate, pH 6.4)/cells plate and were defined as solubilized γ-secretase, as previously shown by Pinnix et al. (2001). Solubilized γ-secretase activity was induced at 37° C. for 2 h with or without the indicated treatments and Aβ 40 generated de novo was quantified by ELISA. Control experiments used the internally quenched fluorogenic γ-secretase substrate NMA-GGWIATVK(DNP)-DRDRDR-NH₂ (λex=355 nm; λem=440 nm) from Calbiochem, which contains the C-terminal β-APP amino acid sequence that is cleaved by γ-secretase and the γ-secretase inhibitor N—[N-(3,5-Difluorophenacetyl-L-alanyl)]-S-phenylglycine t-Butyl Ester (DAPT, Calbiochem).

### Cytotoxicity Assays

Cell viability and cytotoxicity of the tested compounds were routinely assessed using MTT [3-(4,5-dimethylthiazol-2-yl)-2,5-diphenyltetrazolium bromide] assay or relased LDH using the CytoTox 96 Assay according to the manufacturer's instructions (Promega).

### Example 2

### Preparation of 5-(5-(7-(Trifluoromethyl)quinolin-4-ylthio)pentyloxy)-2-(morpholinomethyl)-4H-pyran-4-one dihydrochloride (compound 38)

### 2.1. Preparation of Intermediates 50-52.

2-(Bromomethyl)-5-hydroxy-4H-pyran-4-one 50.

In a 250 mL round-bottomed flask 5-hydroxy-2-(hydroxymethyl)-4H-pyran-4-one (10.0 g, 70.36 mmol) was added to concentrated H₂SO₄ (30 mL). The solution was cooled to 4° C. and bromohydric acid (40 mL) was added dropwise for 1.5 h. HBr vapors were trapped using a NaOH 0.5 N solution. The reaction was stirred at 70° C. for 18.5 h. After cooling ice (220 g) was added with continuous stirring for 1.5 h to obtain a white precipitate. The solid was filtered off and dissolved in ethyl acetate and the solution was dried with MgSO₄, filtered and evaporated. 2-(Bromomethyl)-5-hydroxy-4H-pyran-4-one 50 was obtained as a pale yellow solid (9.1 g, 63% yield).

The structure of compound 50 is presented below:

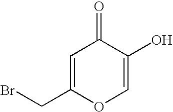

MW: 205.00; Yield: 63%; Pale yellow solid; Mp: 182.5° C.

R_{f}: 0.70 (CH₂Cl₂:MeOH=9:1).

¹H-NMR (CDCl₃, δ): 4.51 (s, 2H, S—CH₂), 4.-8 (s, 2H, Br—CH₂), 7.32-7.45 (m, 5H, Ar—H7.72 (dd, 1H, J=8.8 Hz, J=1.7 Hz, ArH), 8.22 (d, J=8.8 Hz, 1H, ArH), 8.39 (s, 1H, ArH), 8.82 (d, J=4.8 Hz, 1H, ArH).

5-Hydroxy-2-(morpholinomethyl)-4H-pyran-4-one 51.

2-(Bromomethyl)-5-hydroxy-4H-pyran-4-one 50 (5.0 g, 24.4 mmol), morpholine (4.3 mL, 48.8 mmol) and acetonitrile (120 mL) were charged in a 250 ml round-bottomed flask equipped with a magnetic stirrer. The reaction mixture was stirred for 3 h at 80° C. Acetonitrile was evaporated and the residu was extracted with EtOAc (400 mL). The organic layer was washed with water (20 mL), brine (2×20 mL), dried over MgSO₄, filtered and evaporated to dryness. Diethyl ether (25 mL) was added and the product was precipitated and filtered to give after drying 5-hydroxy-2-(morpholinomethyl)-4H-pyran-4-one 51 (4.8 g, 72% yield) as a white solid.

The structure of compound ex 51 is presented below:

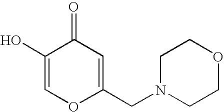

MW: 211.21; Yield: 72%; White solid; Mp: 144.2° C.

R_{f}: 0.37 (CH₂Cl₂:MeOH=95:5).

¹H-NMR (CDCl₃, δ): 2.53 (t, J=4.5 Hz, 4H, N—CH₂), 3.41 (s, 2H, N—CH₂), 3.74 (t, 4H, J=4.5 Hz, O—CH₂), 6.54 (s, 1H, —C═CH), 6.65 (s, 1H, OH), 7.86 (s, 1H, —C═CH).

¹³C-NMR (CDCl₃, δ): 53.4, 59.7, 66.6, 112.2, 138.6, 145.8, 165.2, 174.3.

HPLC: Method A, detection UV 254 nm, 51 RT=1.0 min, peak area 99.5%.

4-(5-Bromopentylthio)-7-(trifluoromethyl)quinoline 52.

7-Trifluoromethyl-4-quinoline-thiol (5 g, 21.8 mmol), 1,5-dibromopentane (23.7 g, 98.1 mmol) and CHCl₃ (60 mL) were charged in a 250 ml round-bottomed flask equipped with a magnetic stirrer. TBABr (0.7 g, 2.2 mmol) and water (40 mL) were added and the reaction mixture was stirred for 48 h at 20° C. The reaction mixture was poured in 100 mL of H₂O with K₂CO₃ (3.0 g, 21.8 mmol) and extracted with CH₂Cl₂ (400 mL). The organic layer was washed with water (30 mL), brine (2×30 mL), dried over MgSO₄, filtered and evaporated at 30° C. to dryness. The crude compound was purified by column chromatography (SiO₂:CH₂Cl₂: MeOH=99.5:0.5 to 98:2) to give after evaporation 4-(5-bromopentylthio)-7-(trifluoromethyl)quinoline 52 (5.9 g, 72% yield) as a white solid.

The structure of compound 52 is presented below:

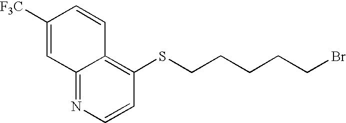

MW: 378.25; Yield: 72%; White solid; Mp: 60.3° C.

R_{f}: 0.75 (CH₂Cl₂: EtOAc=9:1).

¹H-NMR (CDCl₃, δ): 1.67-1.75 (m, 2H, CH₂), 1.81-1.99 (m, 4H, CH₂), 3.14 (t, J=7.2 Hz, 2H, S—CH₂), 3.44 (t, J=6.6 Hz, N—CH₂), 7.25 (d, J=4.8 Hz, 1H, Ar—H), 7.71 (dd, J=8.8 Hz, J=1.8 Hz, 1H, Ar—H), 8.23 (d, J=8.8 Hz, 1H, Ar—H), 8.36 (s, 1H, Ar—H), 8.79 (d, J=4.8 Hz, 1H, Ar—H).

MS-ESI m/z (rel. Int.): 378.0/379.8 ([MH]⁺).

HPLC: Method A, detection UV 254 nm, 52, RT=6.0 min, peak area 99.5%.

2.2. Preparation of 5-(5-(7-(Trifluoromethyl)quinolin-4-ylthio)pentyloxy)-2-(morpholinomethyl)-4H-pyran-4-one dihydrochloride (38).

5-Hydroxy-2-(morpholinomethyl)-4H-pyran-4-one 51 (2.5 g, 12.0 mmol), Cs₂CO₃ (3.9 g, 12.0 mmol) and anhydrous DMF (40 mL) were charged in a 250 ml round-bottomed flask equipped with a magnetic stirrer under inert atmosphere. 4-(5-Bromopentylthio)-7-(trifluoromethyl)quinoline 52 (3.8 g, 10.0 mmol) and NaI (0.2 g, 1.3 mmol) were added and the reaction mixture was stirred for 2 h at 90° C. After evaporation of DMF, the reaction mixture was poured in 50 mL of H₂O, extracted with CH₂Cl₂ (2×200 mL). The organic layer was washed with brine (2×20 mL), dried over MgSO₄, filtered and evaporated to dryness. The crude compound was purified by column chromatography (SiO₂; CH₂Cl₂:MeOH=99:1 to 94:6) to give after evaporation a pure solid (4.4 g, 88% yield). The compound was dissolved in EtOH (150 mL), then HCl 1M in EtOH (22 mL, 21.6 mmol) was added and the reaction mixture was stirred for 2 h at 20° C. After evaporation of EtOH, the hydrochloride compound was dried under vacuum and crystallized in ethanol (50 mL) to yield to compound 38 (3.35 g, 58% yield) as a white powder.

The structure of compound 38 is presented below:

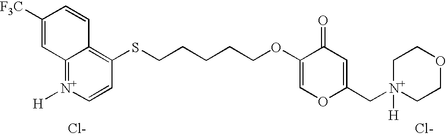

MW: 581.47; Yield: 58%; White powder, Mp: 217.7° C.

R_{f}: 0.40 (CH₂Cl₂:MeOH=95:5).

¹H-NMR (CDCl₃, δ): 1.62-1.64 (m, 2H, CH₂), 1.76-1.87 (m, 4H, CH₂), 3.10-3.38 (m, 4H, N—CH₂), 3.42 (t, J=6.6 Hz, 2H, S—CH₂), 3.85-3.91 (m, 6H, O—CH₂), 4.34 (s, 2H, N—CH₂), 6.77 (s, 1H, C═CH), 7.88 (d, J=5.0 Hz, 1H, Ar—H), 8.08 (d, J=8.9 Hz, 1H, Ar—H), 8.21 (s, 1H, C═CH), 8.45 (d, J=8.8 Hz, 1H, Ar—H), 8.67 (s, 1H, Ar—H), 9.06 (d, J=5.5 Hz, 1H, Ar—H), 11.15 (s, 1H, NH⁺), 12.25 (s, 1H, NH⁺).

MS-ESI m/z (rel. Int.): 509.0 ([MH]⁺, 100).

HPLC: Method A, Detection UV 254 nm, 38 RT=4.40 min, peak area 99.9%.

Anal. (C₂₅H₂₇F₃N₂O₄S.2HCl); C, H, N, S: calcd, 51.64, 5.03, 4.82, 5.51, found, 51.18, 5.06, 4.83, 5.31.

### Example 3

### Compounds of Formula (I) Inhibit Rac1 Activation

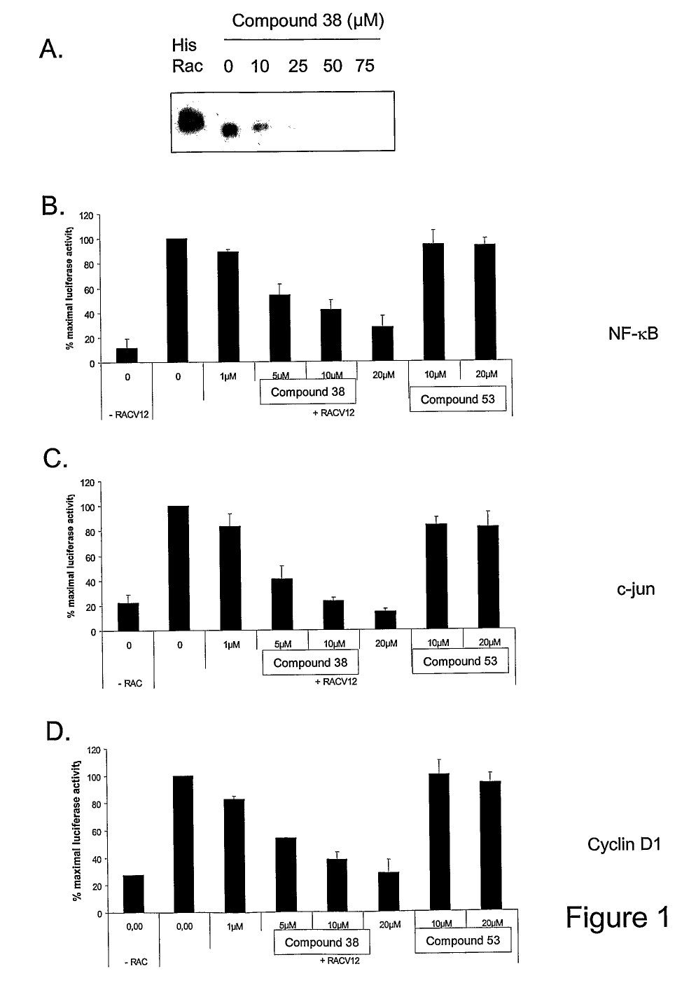

To test whether compounds of formula (I) might affect Rac1 activity, U87-MG cells were treated in different concentrations with compound 38. We used a GST-fusion protein containing the p21-binding domain (PBD) of human p21-activated kinase 1 (Pak1) to affinity precipitate endogenous active Rac1 (GTP-Rac1) from cell lysates to monitor the activation of the small GTPase Rac1. The GST-Pak-PBD fusion protein was incubated with cell lysate and the effector pulled-down active or GTP-Rac1 was detected by Western blot analysis using a specific Rac1 antibody. As shown in FIG. 1A, compound 38 strongly inhibited Rac1 activation in dose-response, leading to more than two times reduction in active Rac1 levels at 10 μM and undetectable levels of active Rac1 levels at 50 μM.

It has been reported that Rho family members can drive transcription from reporter constructs Gal-4-c-Jun plus 5× Gal-4-Luc, HIV-Luc bearing NF-KB binding sites and cyclin D1-Luc. We cotransfected the constitutively active mutant Rac1-Val12 (RACV12) in NIH3T3 cells to probe the effect of compound 38. As shown in FIG. 1, RACV12 elicited transcriptional responses from c-Jun, NF-KB and cyclin D1 reporter constructs. Fold inductions for c-Jun, NF-KB and cyclin D1 reporter constructs were 5, 15 and 4, respectively. Treatment of cells with compound 38 dose-dependently reduced luciferase activity from all 3 reporter constructs and for each, the 50% inhibition dose came at ˜5 μM. These results demonstrated that compound 38 is able to inhibit cellular Rac1 activation and Rac1-mediated transduction pathways. We also tested a control molecule, 5-(5-(Quinazolin-4-yloxy)pentyloxy)-2-((4-methylpiperazin-1-yl)methyl)-4H-pyran-4-one (compound 53), which is totally unable to inhibit Rac1 activation (data not shown). As expected, compound 53 did not change luciferase activity from all 3 reporter constructs.

Dissociation constants (Kd) for compound 38 were determined using fluorescence anisotropy measurement. Fluorescent anisotropy measures the tumbling rate of a fluorophore as a reporter for the size of the fluorophore-complex (compound 38-Rac). Methods used are known to those skilled in the art (Jasuja R, Ramaraj P, Mac R P, Singh A B, Storer T W, Artaza J, Miller A, Singh R, Taylor W E, Lee M L, Davidson T, Sinha-Hikim I, Gonzalez-Cadavid N, Bhasin S., J Clin Endocrinol Metab. 2005, 90(2):855-63; Deprez E, Barbe S, Kolaski M, Leh H, Zouhiri F, Auclair C, Brochon J C, Le Bret M, Mouscadet J F., Mol. Pharmacol. 2004, 65(1):85-98.; Stricher F, Martin L, Barthe P, Pogenberg V, Mechulam A, Menez A, Roumestand C, Veas F, Royer C, Vita C., Biochem J. 2005, 390(Pt 1):29-39). In this study, the intrinsic fluorescence of compound 38 was used. Recombinant Rac proteins were titrated into a solution containing 1 μM compound 38. Anisotropy values Rac-bound compound 38 were determined at graded Rac concentrations using λₑₓ=360 nm; λₑₘ=410 nm. Kds were subsequently determined and are displayed in the table below.

| Recombinant protein | Kd | |
|---|---|---|
| Rac1 | 40 | nM |
| Rac1b | 52 | nM |
| Rac2 | 60 | nM |
| Rac3 | 270 | nM |

These data clearly and unambiguously show that compound 38 inhibits Rac1, as well as Rac1 B and Rac2 and, although to a less extent, Rac3.

### Example 4

### Compounds of Formula (I) Prevent Aβ 40 and Aβ 42 Production In Vitro

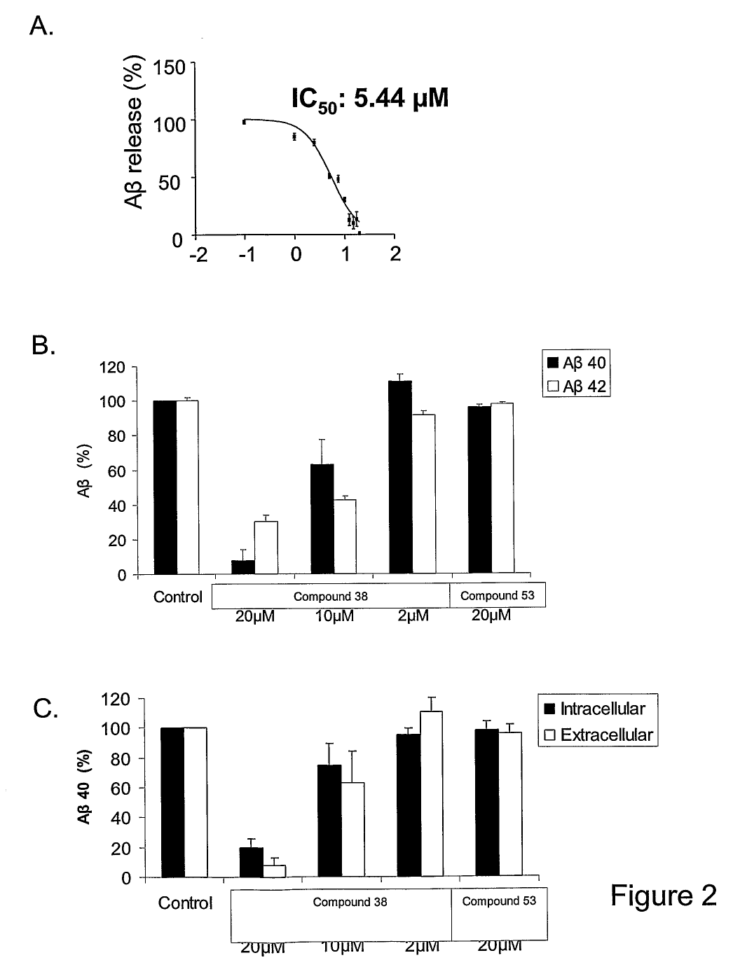

We have examined the effect of compound 38 and compound 53 on the production of Aβ 40 using SH-SY5Y cells endogenously expressing wtAPP. Cells were allowed to secrete in 5 ml medium for 7 hours in the presence of phosphoramidon. Total amount of secreted Aβ 40 in untreated samples was 21.4±3.9 pg/ml and Aβ 42 levels were below the detection limit of the ELISA assay, as reported by others. Incubation of cells for 18 h with 20, 10 or 2 μM compound 38 resulted in 79.8, 30.1 and 12.9% reduction in secreted Aβ 40 levels, respectively. In contrast, compound 53 was strictly inactive in reducing Aβ levels. We calculated compound 38 IC₅₀ as being 5.44 μM using the Prizm Software (FIG. 2 A).

Human APP harbouring the “Swedish” mutation (swAPP) is more prone to processing than wtAPP. To test whether similar inhibition of Aβ levels was also observed following swAPP processing, we used swAPP-HEK293 cells which secrete high quantities of Aβ 40 and of the more amyloidogenic peptide Aβ 42. Aβ 40 and Aβ 42 released in the conditioned medium were quantified after 18 h treatment with 20, 10 or 2 μM compound 38. As reported previously, the total amount of secreted Aβ 40 was approximately 10-fold higher than the total amount of secreted Aβ 42. FIG. 2 B shows that compound 38 dose-dependently inhibits both Aβ species with a similar activity at all three concentrations tested and suggest that inhibition of Aβ peptide occurs independently of the wt or “Swedish” mutation conditions. At 50 μM or above, compound 38 led to Aβ 40 and Aβ 42 levels below the detection limit of the ELISAs tests. Compound 53 was strictly inactive in reducing Aβ 40 and Aβ 42 levels in swAPP-HEK293 cells.

It is now demonstrated that there are two cellular pools of Aβ, both intracellular and extracellular (secreted), that behave independently of one another. We then determined cell-associated Aβ levels as an indication of the effect of compound 38 on intracellular Aβ. We harvested cells in CelLytic-M buffer and assayed cell lysates for levels of intracellular Aβ 40 and 42 species by specific ELISAs. As observed for secreted Aβ 40 and Aβ 42, compound 38 treatment caused a dose-dependent decrease in both Aβ 40 and Aβ 42 intracellular levels after normalization of Aβ levels to cellular protein content (FIG. 2 C). compound 53 was strictly inactive in reducing both intracellular and intracellular Aβ 40 and Aβ 42 levels in swAPP-HEK293 cells (data not shown). Thus, our results thus far indicated that compound 38 induced a decrease in APP processing and subsequent Aβ generation. Furthermore, in both SH-SY5Y and HEK293 cells, cell viability was unaffected by compound 38 or compound 53 as measured by both lactate dehyrodgenase (LDH) assay and MTT [3-(4,5-dimethylthiazol-2-yl)-2,5-diphenyltetrazolium bromide] assays (data not shown).

### Example 5

### Compounds of Formula (I) do not Affect BACE and α-Secretase Pathways

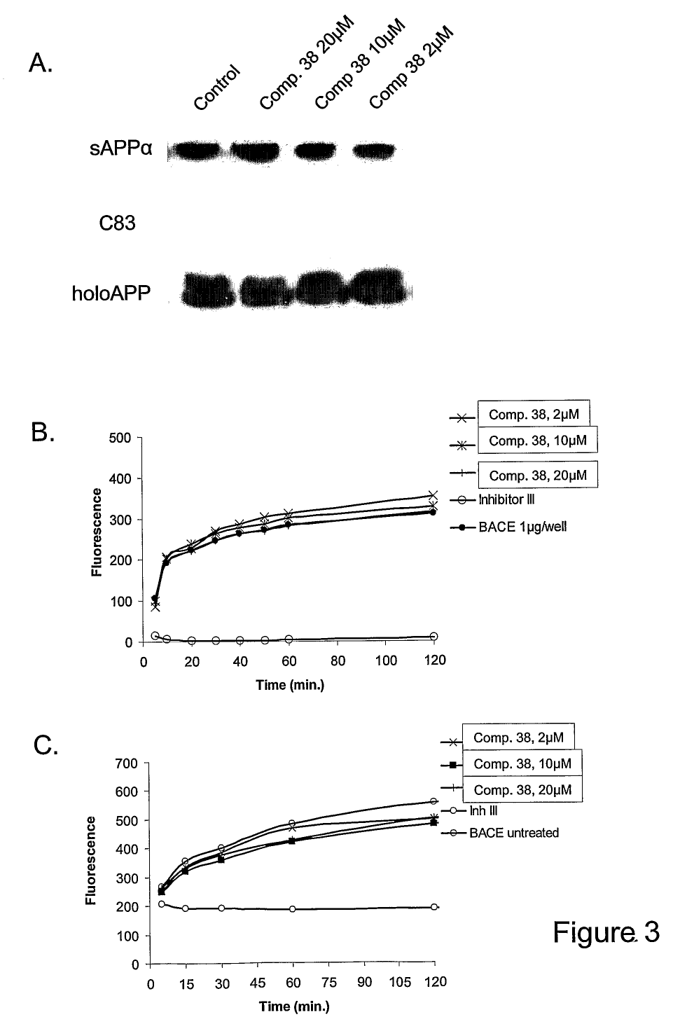

Previous studies have shown that α- and β/γ-pathways may compete for APP substrate under certain conditions. Therefore, increased sAPPα levels may explain decreased Aβ levels. Thus, we tested the effect of compound 38 on the α-secretase pathway by monitorting levels of sAPPα secreted in the culture medium of swAPP-HEK293 and its intracellular counterpart C83. The antibody used here for sAPPα recognizes the last 16 residues of sAPPα that are not present in sAPPP. C83 antibody was detected using anti-APP (AA737-751) antibody. As shown in FIG. 3 A, exposure to 20, 10 or 2 μM compound 38 neither affected sAPPα nor C83 levels, establishing the absence of direct or indirect modulation of α-secretase activity by compound 38. The level of full-length cytosolic and membranous APP was also found to be unchanged on treatment with compound 38, excluding the event of altered APP holoprotein expression, maturation or trafficking.

We then tested the effect of compound 38 on BACE1 activity, the rate-limiting enzyme in Aβ production. To rule out a direct inhibitory effect on BACE activity, a BACE-specific fluorogenic assay was implemented using recombinant human BACE protein diluted at 1 μg/well in 0.1M Na Acetate buffer, pH4.4, which cleaved the quenched fluorogenic Substrate V containing the Swedish mutation, resulting in increased fluorescence. The BACE substrate analog inhibitor III abolished the cleavage, while incubation of compound 38 at 2, 10 or 20 μM (FIG. 3 B) as well as higher concentrations (data not shown) did not affect fluorescence, establishing that compound 38 is unable to affect BACE activity. Next, we tested whether compound 38 could indirectly affect cellular BACE activity using HEK293 cells stably expressing BACE1. BACE activity was present in homogenate proteins from transfected cells. In contrast, very low substrate activity was detected in control cells (data not shown). The activity was inhibited by the well characterized BACE Inhibitor III and incubation of cells for 24 hours with compound 38 at 2, 10 or 20 μM did not affect fluorescence (FIG. 3 C) suggesting that there is no indirect effect of compound 38 on BACE activity. In fact, immunofluorescence studies aimed at determining the subcellular and membranous localization of BACE using antibodies raised against Nter (AA46-65) or Cter (M485-501) parts of BACE in the presence or absence of Triton failed to detect any changes in BACE immunoreactivity following compound 38 treatment (data not shown), suggesting no effect of compound 38 on BACE subcellular localization as well.

### Example 6

### Compounds of Formula (I) Target γ-Secretase Activity

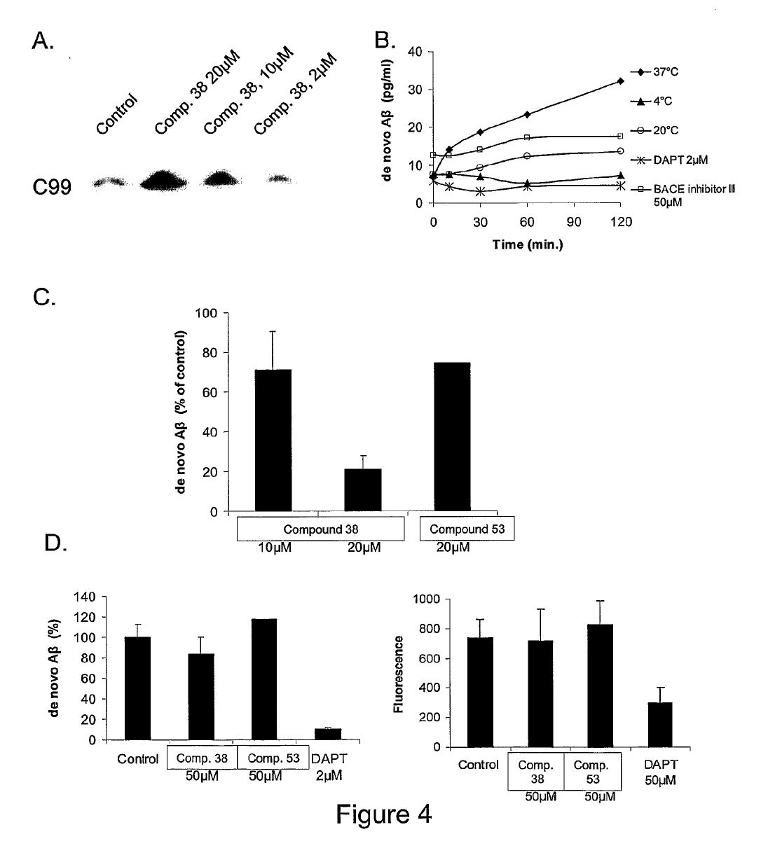

Despite the absence of effect on holoAPP and sAPPα, compound 38 treatment caused a dose-dependent increase in C99 (FIG. 4 A), which is indicative of an inhibitory effect on γ-secretase.

To test this hypothesis, we used an established γ-secretase assay allowing de novo Aβ generation in vitro, using cell membranes as the source of γ-secretase. Solubilized γ-secretase fractions are activated in a γ-secretase buffer (see experimental procedures) and activity is monitored following either the cleavage of an internally quenched fluorogenic γ-secretase substrate, or endogenous C99 itself. The well characterized cell permeable γ-secretase inhibitor DAPT (Lanzn, T. A., Himesn, C. S., Pallanten, G., Adamsn, L., Yamazakin, S., Amoren, B., and Merchantn, K. M. (2003) J. Pharmacol. Exp. Ther. 305, 864-871) is included as control. C99 cleavage by γ-secretase was measured by de novo Aβ 40 generation. De novo Aβ 40 production increased with time and was optimal at 37° C. (FIG. 4 B). Longer incubation times (up to 5 hours) showed that Aβ 40 reached a plateau, probably due to substrate depletion (data not shown). When temperature decreased, Aβ 40 generation decreased in parallel and was abolished at 4° C., in accordance with an enzymatic processing. Other control studies showed that Aβ 40 generation was sensitive to denaturing agents such as boiling the samples at 95° C. (data not shown). Aβ production was completely inhibited by the γ-secretase inhibitor DAPT at 2 μM and by the BACE inhibitor II. These data confirmed that de novo Aβ 40 generation resulted from C99 cleavage by γ-secretase, C99 being generated from swAPP by BACE. These results also suggested that preexisting Aβ associated to the membrane did not significantly participate to the measurement of de novo generated Aβ 40. Incubation of cells with compound 38 for 16 hours resulted in a dose-dependent reduction in de novo Aβ production from C99 (FIG. 4 C), consistent with an action on γ-secretase activity. In contrast, compound 53 did not affect γ-secretase activity at any of the concentrations tested.

To test whether compound 38 could act as a direct γ-secretase inhibitor, compound 38 was added to solubilized γ-secretase preparations obtained from untreated cell. At 50 μM, compound 38 did not change de novo Aβ 40 generation from membrane preparations (FIG. 4 D-left graph). Similar lack of effect was seen with concentrations as high as 4 mM (not shown) and this result was confirmed using the fluorogenic γ-secretase substrate which contains the C-terminal β-APP amino acid sequence cleaved by γ-secretase (FIG. 4 D-right graph), establishing that compound 38 is not a direct competitive inhibitor of γ-secretase. Rather, our results suggest that in cells, compound 38 negatively impacts γ-secretase activity so that Aβ production pathway is blocked.

### Example 7

### Compounds of Formula (I) do not Inhibit Notch-1 Cleavage

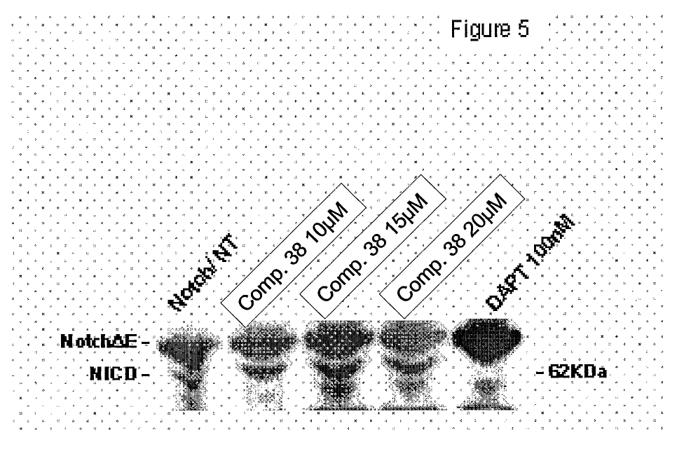

Many γ-secretase inhibitors also inhibit the cleavage of the γ-secretase substrate Notch-1, the signalling of which is required in the adult organism for ongoing differentiation processes of the immune system and the gastrointerstinal tract. In contrast, agents that modulate γ-secretase activity such as NSAIDs or Gleevec and reduce Aβ 42 levels do not inhibit Notch-1 cleavage. To determine whether compound 38 inhibits Notch cleavage or not, we used Hela cells, which endogenously present high γ-secretase activity as compared to other cell types (Takahashi, Y., Hayashi, I., Tominari, Y., Rikimaru, K., Morohashi, Y., Kan, T., Natsugari, H., Fukuyama, T., Tomita, T., and Iwatsubo, T. (2003) J. Biol. Chem. 278, 18664-18670). Hela cells were transiently transfected to overexpress N-terminally truncated Notch-1 (NotchΔE) and exposed for up to 16 hours various concentrations of compound 38 or of the γ-secretase inhibitor DAPT (2 μM). Detection of NotchΔE and the γ-secretase cleavage product NICD by Western blot showed that compound 38 did not affect Notch cleavage at any concentration tested (FIG. 5). In contrast, Notch cleavage was potently inhibited by DAPT leading to virtually undetectable NICD levels.

### Example 8

### Compounds of Formula (I) Prevent Aβ 40 and Aβ 42 Production In Vivo

The effects of compound 38 were tested in the guinea pig to determine whether the observed reductions in Aβ 40 and Aβ 42 observed in cell lines overexpressing wild-type and human mutant APP can be reproduced in vivo. We used normal wild-type albino guinea pigs as a model, because guinea pigs are an established model for physiological APP processing and Aβ production. In addition, their Aβ 40 and Aβ 42 peptides are identical to human Aβ and can be readily detected by the Biosource sandwich ELISA.

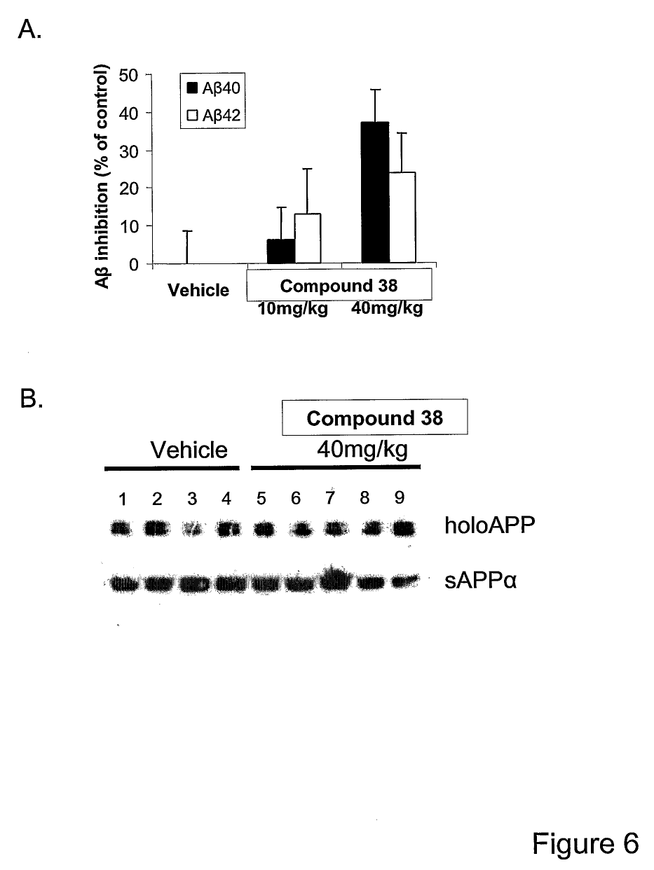

Preliminary experiments performed in rats (V.P., personal communication) showed that compound 38 displays good tolerability. In particular, the compound showed no genotoxicity (Ames test) and acute toxicity in rat showed a LD50 above 1000 mg/kg or at 50 mg/kg (p.o. administration or i.v. administration, respectively). More important, brain concentrations of compound 38 could be determined for 100 mg/kg, p.o. or 5 mg/kg, i.v. (54 ng/g and 130 ng/g, respectively), suggesting that compound 38 was able to cross the blood brain barrier. We opted for a straightforward delivery mode in Guinea pigs and delivered compound 38 over 15 days by means of daily intraperitoneal injections, at two concentrations (10 and 40 mg/kg). We used a guanidine-based extraction protocol to ensure recovery of both triton-soluble and triton insoluble Aβ fractions. In control animals, recovered Aβ 40 concentration was 627.9 pg/ml. compound 38 (40 mg/kg per day) lowered brain Aβ 40 by 37% with (p<0.05 by Wilcoxon test) (FIG. 6 A). For Aβ 42, a high variability in measurement was observed, probably due to the smaller amounts of peptide. The same dose of the compound compound 38 (40 mg/kg per day) caused 23.6% decrease in Aβ 42 levels, which was however just below significance by Wilcoxon test. No significant changes in C83 and C99 levels were detected in treated animals (FIG. 6 B), with no change in full-length APP levels, consistent with an inhibitory action at the level of APP cleavage. No obvious signs of behavioral or anatomic abnormalities were observed for any of the treated animals at the indicated doses.

### Example 9

### Compound 49 Prevents Aβ 40 and Aβ 42 PRODUCTION in Vitro without Affecting Notch and sAPPα

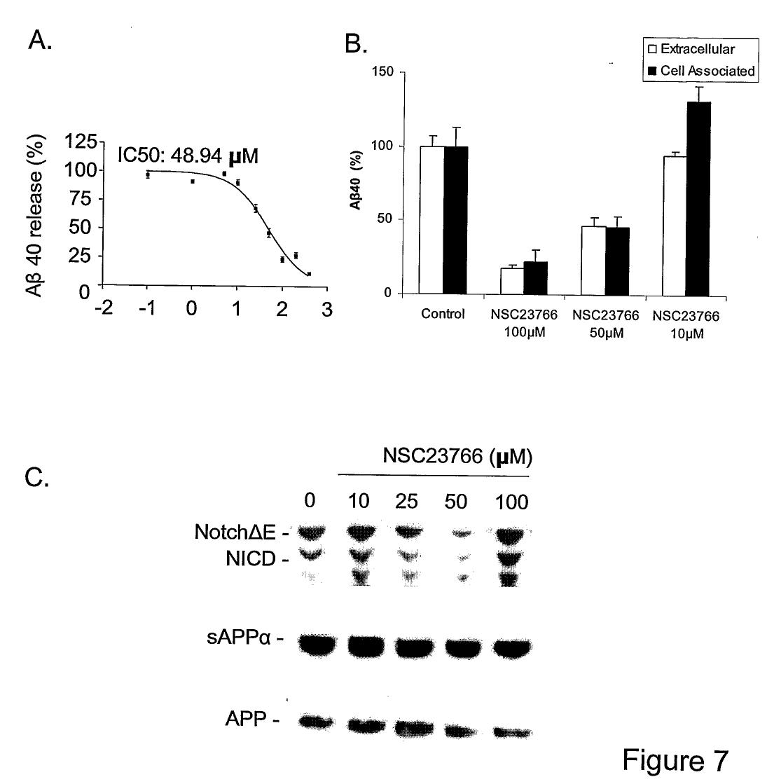

Gao et al. (2004) described NSC23766, a cell-permeable Rac1-specific inhibitor with an IC₅₀˜50 μM, which was shown not to affect the activity of Cdc42 or RhoA (Gao, Y., Dickerson, J. B., Guo, F., Zheng, J., and Zheng, Y. (2004) Proc. Natl. Acad. Sci. USA 101, 7618-7623). We therefore used NSC23766 (compound 49) to determine whether we could recapitulate the effect of compound 38 on APP processing and Aβ production. On swAPP-HEK293 cells, treatment with various concentrations of compound 49 dose-dependently reduced secreted Aβ 40 levels (FIG. 7 A). Interestingly, the IC₅₀ was 48.94 μM, in line with its reported effects on Rac1 inhibition. Based on IC₅₀ determination, compound 38 is 10-fold more potent than compound 49. As for compound 38, we found no intracellular accumulation of Aβ 40 (FIG. 7 B), showing again that APP processing was impaired. Accordingly, both extracellular and intracellular levels of Aβ 42 were also dose-dependently decreased, with 57.97% inhibition of released Aβ 42 at the concentration of 50 μM. Up to 400 μM, we found no evidence of cytotoxity measured by both LDH and MTT assays (data not shown), suggesting that decreased Aβ levels were not due to cell viability impairment and that Rac1 inhibition itself is not deleterious for cell survival.

Finally, we tested the effect of compound 49 on APP, C83, sAPPα and NotchΔE/NICD in swAPP-HEK293 and pSC2+ΔE3MV-6MT transfected Hela cells, respectively treated for 16 hours with the indicated concentrations of compound 49 and found no alterations in APP expression and maturation, α-secretase pathway or Notch processing (FIG. 7 C).

In conclusion, our data indicated that compound 49 recapitulated the effects of compound 38 by preventing Aβ 40 and Aβ 42 production in vitro without affecting Notch and sAPPα.

### Example 10

### Discussion

The present invention shows that two structurally different Rac1 inhibitors, compound 38 and the commercially available compound 49, modulate APP processing, diverting APP away from γ-secretase cleavage without directly acting as γ-secretase inhibitors. Our data suggest that γ-secretase processing of APP is under a positive control by Rac1 and that the consequence of Rac1 inhibition is decreased intracellular and extracellular levels of Aβ 40 and 42 peptides.

Compound 38 belongs to a new family of chemical entities inhibiting Rac1 (Leblond et al., submitted) and Rac1-depedent cytoskeleton rearrangements and is selective over cdc42 and RhoA (Picard et al., submitted). Compound 49, which does not interact with cdc42 and RhoA, mimics the effect of compound 38 on Aβ production. Interestingly, compound 49 and compound 38 do not appear to act similarly on Rac1 activation, as compound 49 is not able to inhibit activated L61Rac1 (Gao et al., (2004) Proc. Natl. Acad. Sci. USA 101, 7618-7623) as well as activated Rac1V12 (our unpublished observations) mutants, in contrast to compound 38. The fact that compound 38 interacts with, and inhibits Rac1, as demonstrated with effector PAK1 pull-down assay, and that compound 38 also blocks the downstream signalisation events induced by Rac1V12 mutant strongly suggests that compound 38 elicits its effects on Aβ production by inhibiting Rac1. In support of this hypothesis, compound 38 prevents Rac1 activation by its GEFs in exchange assay as well as Rac1-dependent cytoskeleton rearrangements and angiogenesis (Picard et al., submitted).

In conclusion, the present invention provide in vitro and in vivo evidence of a new therapeutic approach to Alzheimer's disease offered by the selective inhibition of the small GTPase protein Rac1 which does not affect Notch processing. Our data suggest that Rac1 inhibition may interfere with APP processing by decreasing the likelihood of γ-secretase cleavage occurrence. In vivo, compound 38 provides ˜30% reduction in Aβ levels. Interestingly, patients with early-onset AD who have mutations in APP or presenilins present Aβ42 levels that are increased by as little as 30% (Scheuner, D. et al. 1996. Nat. Med. 2:864-870). Studies of these same mutations in transgenic mice indicate that these small increases in Aβ42 levels markedly accelerate AR deposition (Duff, K. et al. 1996. Increased amyloid-beta42(43) in brains of mice expressing mutant presenilin 1. Nature. 383:710-713). Therefore, given the progressive nature of the disease, a small (20-30%) decrease in Aβ production shall have a profound impact on AD by lowering Aβ levels below concentrations responsible for neurodegeneration.

1-15. (canceled)

16. A method of treating an amyloïd beta peptide-related disorder in a mammalian subject, comprising administering to a subject in need thereof an amount of a Rac1 inhibitor effective at reducing APP processing in said subject.

17. A method of inhibiting the generation of an amyloïd beta peptide in a mammalian subject, comprising administering to a subject in need thereof an amount of a Rac1 inhibitor effective at reducing APP processing in said subject.

18. A method of claim 16, wherein the compound further inhibits Rac1B and/or Rac2 and/or Rac3.

19. A method of claim 16, wherein the compound does not substantially alter Notch cleavage or BACE activity.

20. A method of claim 16, wherein the Rac1 inhibitor is a compound of formula (I): 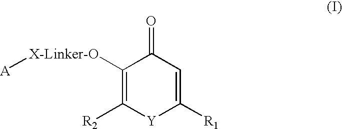
wherein:
R₁ is selected from the group consisting of: 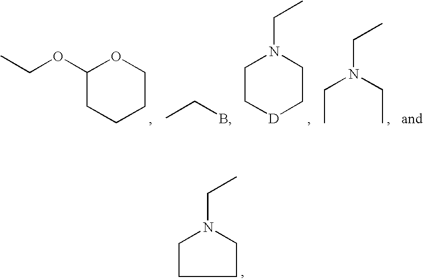
R₂ represents a hydrogen atom, an alkyl or alkenyl group containing from 3 to 6 carbon atoms;
B represents an halogen atom, preferably chlorine, a hydroxyl group, a —O—CH₂—O—CH₃ (MOM) group, a —O—CH₂—O—CH₂—CH₂—O—CH₃ (MEM) group, a —OSO₂-alkyl group or a —OSi(CH₃)₂tBu;
D represents an oxygen atom, NR₃, CR′R″ or a sulfur atom;
X represents an oxygen atom, a sulfur atom or a radical —NR₄—;
Y represents an oxygen atom, a sulfur atom or a radical —NR₄—;
R₃ represents a hydrogen, an alkyl group, a carboxylate group, an acyl group, a carboxamide group or a SO₂-alkyl group;
R′ and R″, identical or different, represent a hydrogen atom or an alkyl radical;
R₄, identical or different, is selected from a group consisting of a hydrogen atom, an alkyl group having from 1 to 10 carbon atoms, an aryl and an aralkyl;
“linker” represents (CH₂)ₙ, wherein n represents an integer between 1 and 10 inclusive, optionally interrupted by an heteroatom (preferably N, O, S and P) or a carbonyl group, or an aryldialkyl (preferably xylenyl) group
A represents a group selected from: 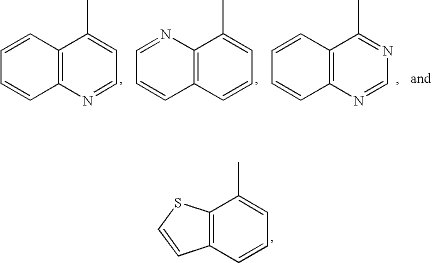
optionally A is substituted,
its tautomers, optical and geometrical isomers, racemates, salts, hydrates and mixtures thereof.

21. The method of claim 20, wherein, in formula (I):
X is sulfur, —NH— or oxygen; and/or
Y is oxygen; and/or
“linker” represents (CH₂)ₙ, wherein n is from 2 to 9, preferably 4 to 7, inclusive, or the meta, ortho or para-xylenyl groups, —CH₂CH₂OCH₂CH₂— and —(C═O)CH₂CH₂CH₂CH₂—); and/or
R₁ is 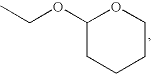
—CH₂N(Et₂) and —CH₂pyrrolidine, 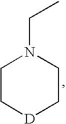
wherein D is oxygen, sulfur, —CH₂— or NR₃, wherein R₃ preferably represents H or an alkyl group (said alkyl is more specifically a methyl radical), and —CH₂—B, wherein B is a —O—CH₂—O—CH₃ group or —OSO₂-alkyl group (wherein alkyl is preferably methyl) or halogen (preferably chlorine of fluorine); and/or
R₂ is a hydrogen atom; and/or
A is a substituted group as defined above.

22. The method of claim 20, wherein, in formula (I):
X is sulfur; and/or
Y is oxygen; and/or
“linker” represents (CH₂)ₙ, wherein n is from 4 to 7, inclusive; and/or
R₁ is 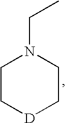
wherein D is oxygen, and/or
R₂ is a hydrogen atom; and/or
A is a group of formula 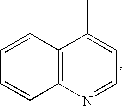
optionnally substituted, most preferably by a trifluoro(C₁-C₆)alkyl group, particularly the CF₃ group.

23. The method of claim 20, wherein the compound is selected from the group consisting of:
2-(Tetrahydro-pyran-2-yloxymethyl)-5-[5-(7-trifluoromethyl-quinolin-4-yloxy)-pentyloxy]-pyran-4-one (1)
5-[5-(6-Fluoro-2-methyl-quinolin-4-yloxy)-pentyloxy]-2-(tetrahydro-pyran-2-yloxymethyl)-4H-pyran-4-one (2)
5-[5-(6-Fluoro-2-trifluoromethyl-quinolin-4-yloxy)-pentyloxy]-2-(tetrahydro-pyran-2-yloxymethyl)-4H-pyran-4-one (3)
5-[5-(7-Propyl-quinolin-8-yloxy)-pentyloxy]-2-(tetrahydro-pyran-2-yloxymethyl)-4H-pyran-4-one (4)
5-[5-(Benzo[b]thiophen-7-yloxy)-pentyloxy]-2-(tetrahydro-pyran-2-yloxymethyl)-4H-pyran-4-one (5)
2-(Tetrahydro-pyran-2-yloxymethyl)-5-[5-(7-trifluoromethyl-quinolin-4-ylsulfanyl)-pentyloxy]-4H-pyran-4-one (6)
2-(Tetrahydro-pyran-2-yloxymethyl)-5-[4-(7-trifluoromethyl-quinolin-4-ylsulfanyl)-butoxy]-4H-pyran-4-one (7)
2-(Tetrahydro-pyran-2-yloxymethyl)-5-[6-(7-trifluoromethyl-quinolin-4-ylsulfanyl)-hexyloxy]-4H-pyran-4-one (8)
2-Hydroxymethyl-5-[5-(7-trifluoromethyl-quinolin-4-ylsulfanyl)-pentyloxy]-4H-pyran-4-one hydrochloride salt (9)
2-Hydroxymethyl-5-[5-(7-trifluoromethyl-quinolin-4-ylsulfanyl)-pentyloxy]-4H-pyran-4-one (10)
2-Methoxymethoxymethyl-5-[5-(7-trifluoromethyl-quinolin-4-ylsulfanyl)-pentyloxy]-4H-pyran-4-one (11)
2-Chloromethyl-5-[5-(7-trifluoromethyl-quinolin-4-ylsulfanyl)-pentyloxy]-4H-pyran-4-one (12)
2-(4-Methyl-piperazin-1-ylmethyl)-5-[5-(7-trifluoromethyl-quinolin-4-ylsulfanyl)-pentyloxy]-4H-pyran-4-one (13)
2-Morpholin-4-ylmethyl-5-[5-(7-trifluoromethyl-quinolin-4-ylsulfanyl)-pentyloxy]-4H-pyran-4-one (14)
5-(5-(7-(Trifluoromethyl)quinolin-4-ylthio)pentyloxy)-2-(fluoromethyl)-4H-pyran-4-one (15)
5-(5-(7-(Trifluoromethyl)quinolin-4-ylthio)pentyloxy)-2-((piperidin-1-yl)methyl)-4H-pyran-4-one (16)
5-(5-(7-(Trifluoromethyl)quinolin-4-ylthio)pentyloxy)-2-(thiomorpholino-methyl)-4H-pyran-4-one (17)
2-((Diethylamino)methyl)-5-(5-(7-(trifluoromethyl)quinolin-4-ylthio)pentyloxy)-4H-pyran-4-one (18)
4-[5-(6-Morpholin-4-ylmethyl-4-oxo-4H-pyran-3-yloxy)-pentyloxy]-7-trifluoromethyl-quinoline-3-carboxylic acid ethyl ester (19)
5-(5-(8-(Trifluoromethyl)quinolin-4-yloxy)pentyloxy)-2-((4-methylpiperazin-1-yl)methyl)-4H-pyran-4-one (20)
5-(5-(8-(Trifluoromethyl)quinolin-4-yloxy)pentyloxy)-2-(morpholinomethyl)-4H-pyran-4-one (21)
5-(5-(7-(Trifluoromethyl)quinolin-4-yloxy)pentyloxy)-2-(morpholinomethyl)-4H-pyran-4-one (22)
5-(5-(7-(Trifluoromethyl)quinolin-4-yloxy)pentyloxy)-2-((4-methylpiperazin-1-yl)methyl)-4H-pyran-4-one (23)
5-(5-(6-(Trifluoromethyl)quinolin-4-yloxy)pentyloxy)-2-(morpholinomethyl)-4H-pyran-4-one (24)
4-[5-(6-(4-Methyl-piperazin-1-ylmethyl)-4-oxo-4H-pyran-3-yloxy)-pentyloxy]-7-trifluoromethyl-quinoline-3-carboxylic acid ethyl ester (25)
4-[5-(6-(4-Methyl-piperazin-1-ylmethyl)-4-oxo-4H-pyran-3-yloxy)-pentyloxy]-7-trifluoromethyl-quinoline-3-carboxylic acid ethyl ester (26)
5-((4-((7-(Trifluoromethyl)quinolin-4-ylthio)methyl)phenyl)methoxy)-2-(morpholinomethyl)-4H-pyran-4-one (27)
5-((2-((7-(Trifluoromethyl)quinolin-4-ylthio)methyl)phenyl)methoxy)-2-(morpholinomethyl)-4H-pyran-4-one (28)
5-(5-(7-(Trifluoromethyl)quinolin-4-ylthio)pentyloxy)-2-((4-acetylpiperazin-1-yl)methyl)-4H-pyran-4-one (29)
4-((5-(5-(7-(Trifluoromethyl)quinolin-4-ylthio)pentyloxy)-4-oxo-4H-pyran-2-yl)methyl)-N,N-diethylpiperazine-1-carboxamide (30)
5-(5-(7-(Trifluoromethyl)quinolin-4-ylthio)pentyloxy)-2-((4-(pivaloyl)piperazin-1-yl)methyl)-4H-pyran-4-one (31)
4-((5-(5-(7-(Trifluoromethyl)quinolin-4-ylthio)pentyloxy)-4-oxo-4H-pyran-2-yl)methyl)-N,N-di-isopropylpiperazine-1-carboxamide (32)
5-(5-(7-(Trifluoromethyl)quinolin-4-ylthio)pentyloxy)-2-((4-methylsulfonylpiperazin-1-yl)methyl)-4H-pyran-4-one (33)
4-((5-(5-(7-(Trifluoromethyl)quinolin-4-ylthio)pentyloxy)-4-oxo-4H-pyran-2-yl)methyl)-N-tert-butylpiperazine-1-carboxamide (34)
4-((5-(5-(7-(Trifluoromethyl)quinolin-4-ylthio)pentyloxy)-4-oxo-4H-pyran-2-yl)methyl)-N-methylpiperazine-1-carboxamide (35)
5-(6-(Morpholinomethyl)-4-oxo-4H-pyran-3-yloxy)-N-(7-(trifluoromethyl)quinolin-4-yl)pentanamide (36)
5-(2-(2-(7-(Trifluoromethyl)quinolin-4-ylthio)ethoxy)ethoxy)-2-(morpholinomethyl)-4H-pyran-4-one (37)
5-(5-(7-(Trifluoromethyl)quinolin-4-ylthio)pentyloxy)-2-(morpholinomethyl)-4H-pyran-4-one dihydrochloride (38)
5-((3-((7-(Trifluoromethyl)quinolin-4-ylthio)methyl)phenyl)methoxy)-2-(morpholinomethyl)-4H-pyran-4-one dihydrochloride (39)
tert-Butyl 4-((5-(5-(7-(trifluoromethyl)quinolin-4-ylthio)pentyloxy)-4-oxo-4H-pyran-2-yl)methyl)piperazine-1-carboxylate (40)
tert-Butyl 4-((5-(5-(7-chloroquinolin-4-yloxy)pentyloxy)-4-oxo-4H-pyran-2-yl)methyl)piperazine-1-carboxylate (41)
5-(5-(7-(Trifluoromethyl)quinolin-4-ylthio)pentyloxy)-2-((4-methylpiperazin-1-yl)methyl)-4H-pyran-4-one trihydrochloride (42)
5-(5-(7-(Trifluoromethyl)quinolin-4-ylthio)pentyloxy)-2-((piperazin-1-yl)methyl)-4H-pyran-4-one trihydrochloride (43)
5-(5-(7-Chloroquinolin-4-yloxy)pentyloxy)-2-((piperazin-1-yl)methyl)-4H-pyran-4-one (44)
5-(2-(7-(Trifluoromethyl)quinolin-4-ylthio)ethoxy)-2-((tetrahydro-2H-pyran-2-yloxy)methyl)-4H-pyran-4-one (45)
5-(8-(7-(Trifluoromethyl)quinolin-4-ylthio)octyloxy)-2-((tetrahydro-2H-pyran-2-yloxy)methyl)-4H-pyran-4-one (46)
5-(7-(7-(Trifluoromethyl)quinolin-4-ylthio)heptyloxy)-2-((tetrahydro-2H-pyran-2-yloxy)methyl)-4H-pyran-4-one (47)
5-(2-(7-(Trifluoromethyl)quinolin-4-yloxy)ethoxy)-2-(morpholinomethyl)-4H-pyran-4-one (48).

24. The method of claim 16, wherein the compound is compound 38 or its free base.

25. The method of claim 16, wherein the compound is compound 49.

26. The method of claim 16, for treating Alzheimer's disease.

27. A method of treating Alzheimer's disease in a human subject, the method comprising administering to a human subject in need thereof an effective amount of compound of formula: 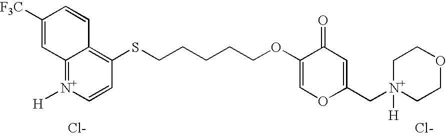
or its free base form or another salt thereof.

28. A method of producing, identifying, selecting or optimising candidate compounds for use in the treatment of amyloïd beta peptide-related disorders, the method comprising determining whether a test compound inhibits Rac1, Rac1 inhibition being an indication that the test compound is a candidate compound for use in the treatment of amyloïd beta peptide-related disorders.

29. The method of claim 28, comprising contacting the test compound and Rac1 and determining whether the compound binds Rac1 or inhibits Rac1-dependent cytoskeleton rearrangements.

30. The method of claim 28, wherein Rac1 inhibition is assessed using the effector PAK1 pull-down assay.
